(19) United States
(12) Patent Application Publication
(10) Pub. No.: US 2018/0143199 A1
(43) Pub. Date: May 24, 2018

(54) METHODS OF IDENTIFYING GLIOBLASTOMA PATIENTS AS SUSCEPTIBLE TO ANTI-ANGIOGENIC THERAPY USING QUANTITATIVE IMAGING FEATURES AND MOLECULAR PROFILING

(71) Applicant: The Board of Trustees of the Leland Stanford Junior University, Palo Alto, CA (US)

(72) Inventors: Ting Liu, Stanford, CA (US); Daniel L. Rubin, Stanford, CA (US)

(21) Appl. No.: 15/821,703

(22) Filed: Nov. 22, 2017

Related U.S. Application Data
(60) Provisional application No. 62/425,999, filed on Nov. 23, 2016.

(51) Int. Cl. G01N 33/574; C12Q 1/6886; G06K 9/62; G06T 7/00; A61B 5/055

(52) U.S. Cl. CPC G01N 33/57407; C12Q 1/6886; G06K 9/62; G06T 7/0002; G06K 2209/05; C12Q 2600/158; G01N 2800/50; G01N 2800/7014; G01N 2800/60; A61B 5/055

(57) ABSTRACT

The present invention provides methods to predict the treatment response of brain tumors such as glioblastoma multiforme to anti-angiogenic therapy based on quantitative perfusion-weighted MRI that can optionally be combined with intra-tumor specific molecular profiling. Since only a subset of brain cancer patients will benefit from anti-angiogenic therapy, identification of this subset is critical so that the effectiveness of the patient's current anti-cancer treatment regimen and the patient's survival likelihood can be increased by the inclusion of an anti-angiogenic agent.

### RELATED APPLICATION

This application claims priority and other benefits from U.S. Provisional Patent Application Ser. No. 62/425,999, filed Nov. 23, 2016, entitled “Quantitative MRI Perfusion Signature For Predicting Treatment Response Of Glioblastoma Multiforme Subtypes To Anti-Angiogenic Therapy.” Its entire content is specifically incorporated herein by reference.

### STATEMENT OF GOVERNMENT SUPPORT

This invention was made with Government support under contracts CA142555, CA190214 and EB020527 awarded by the National Institutes of Health. The Government has certain rights in the invention.

### TECHNICAL FIELD OF THE INVENTION

The present invention relates generally to imaging biomarkers, in particular to imaging biomarkers for predicting treatment response of brain tumor subtypes to anti-angiogenic therapy using quantitative imaging features.

### BACKGROUND

Glioblastoma multiforme (GBM, World Health Organization grade IV) is a high-grade glioma, and the most common and malignant brain cancer in adults. Despite multimodal therapy of surgical resection, radiation, and chemotherapy, relapse occurs frequently and the median survival prospects are generally less than two years (Omuro & DeAngelis, 2013). Studies show that GBM is a heterogeneous disease, reflected by mixed genetic patterns, varied radiographic phenotypes, and disparate clinical outcomes. Thus, defining characteristic phenotypes of GBM that distinguish clinically-relevant subgroups could enable tailoring treatment to these subgroups.

Therapeutic drugs targeting tumor biological processes are being developed and evaluated for their efficacy in improving patient clinical outcomes (Thomas et al., 2014). Recent advances in cancer immunotherapy in mouse models show promising results to potentially identify peptides arising from tumor-specific mutations that may trigger a therapeutic immune response (Yadav et al., 2014). Angiogenesis is a prominent pathophysiological process in GBM that is defined by the formation of new blood vessels to supply nutrients and oxygen to rapidly proliferating tumor cells via up-regulation of vascular endothelial growth factor A (VEGF-A) (Zhang et al., 2014). The anti-angiogenic agent bevacizumab, a humanized monoclonal antibody against VEGF-A to block angiogenesis, was approved for recurrent GBM patients (Kreisl et al., 2009; Friedman et al., 2009).

A subsequent clinical trial evaluating bevacizumab in newly diagnosed GBM patients found no survival advantage of the treatment (Gilbert et al., 2014; Chinot et al., 2014). These patients were assessed as a uniform group with the same clinical diagnosis; however, the fact that GBM is a heterogeneous disease suggests the potential of stratifying patients into subgroups and assessing subgroup-specific responses to anti-angiogenic therapy.

Recent large-scale studies using The Cancer Genome Atlas (TCGA) database have provided a comprehensive genomic, epigenetic, transcriptional, and protein-level characterization of GBM (Brennan et al., 2013; Verhaak et al., 2010), with the ultimate goal of translating this molecular understanding to inform clinical decisions. The integrated analysis of imaging and genomics data is establishing bridges that link our understanding of tissue-level features to molecular counterparts that may help characterize new aspects of disease (Gevaert et al., 2014). A recent study has identified molecular signatures associated with prognostic clusters based on tumor morphological features (Itakura et al., 2015). Another study has found that the tumor location that is associated with poor survival has a distinct molecular profile (Liu et al., 2016).

Biomedical imaging provides morphologic, metabolic and functional information about intact tissues in a spatially and temporally resolved manner. Magnetic resonance imaging is used as the primary modality for the clinical diagnosis of GBM. Prominent imaging features of GBM include heterogeneous enhancement with central necrotic regions on contrast-enhanced T1-weighted image (Omuro & DeAngelis, 2013). Dynamic susceptibility-weighted contrast-enhanced (DSC) perfusion MR imaging is an advanced MR technique that has increasingly become an integral part of the diagnostic workup of GBM (Barajas & Cha, 2014). Whereas T1-weighted imaging shows morphological phenotypes of GBM, perfusion-weighted imaging (PWI) non-invasively detects functional and physiologic phenotypes of tumor vascular characteristics of cancers, allowing indirect assessment of angiogenesis (Barajas & Cha, 2014; Hakyemez et al., 2006). Relative cerebral blood volume (rCBV) quantified from PWI enables voxel-based measurement across the contrast-enhancing lesion (CEL), showing regional microvascular variation that can characterize GBM lesions (Hu et al., 2012; Barajas et al., 2012).

It would be highly desirable to have non-invasive methods available that can serve as imaging biomarkers that also capture the molecular heterogeneity of brain tumors to identify brain tumor patients who are susceptible to anti-angiogenic treatment and to facilitate treatment planning so that a targeted and survival-prolonging treatment approach can be implemented as soon as possible.

### SUMMARY

The present invention provides methods for predicting the susceptibility of a patient who suffers from a brain tumor such as glioblastoma to anti-angiogenic therapy based on brain tumor subtypes using quantitative perfusion imaging features that provide a phenotypic characterization of blood perfusion both of the tumor and of tumor heterogeneity. Optionally, these quantitative imaging features can be combined with genomic data obtained from gene expression or protein expression analysis to characterize brain tumor subtypes on a perfusion phenotypic as well as molecular basis. If, e.g., a patient suffering from glioblastoma is found to be susceptible to anti-angiogenic therapy, then the inclusion of an anti-angiogenic agent to the patient's current anti-cancer treatment regimen will likely increase the effectiveness of the treatment regimen and prolong the patient's survival.

In a first aspect, the present invention provides a computer-implemented method for non-invasively identifying a subject suffering from a brain tumor as susceptible to anti-angiogenic therapy comprising determining quantitative dynamic susceptibility contrast (DSC) T2* perfusion-weighted image features from tissue of said brain tumor to determine said subject's tumor phenotypic angiogenic profile, and comparing said subject's tumor phenotypic angiogenic perfusion profile with a reference phenotypic angiogenic tumor perfusion profile, wherein a deviating profile for said subject relative to said reference profile identifies said subject as susceptible to anti-angiogenic therapy. In an additional step, said subject's tumor phenotypic angiogenic perfusion profile can be further defined with a molecular profile obtained from gene expression or protein expression analysis to create a phenotypic perfusion and molecular tumor angiogenic profile from said subject which is then compared to a reference phenotypic perfusion and molecular tumor angiogenic profile, wherein a deviating profile for said subject relative to said reference profile identifies said subject as susceptible to anti-angiogenic therapy.

In one embodiment of the present invention, the brain tumor is glioblastoma. In some embodiments, the quantitative image features are determined by measuring perfusion-weighted image data using magnetic resonance imaging to quantify regional variation and intra-tumor heterogeneity. In some embodiments, these features include, but are not limited to, mean, median, variance, maximum, skewness, kurtosis, 20 histogram bins of perfusion voxel values within the tumor region from rCBV values ranging from 0.5 to 10 at an increment of 0.5, and 20 perfusion elevated features quantifying elevated perfusion tumor burden, which is the fraction of the tumor with rCBV voxel values greater than a threshold (rCBV_{elevated}), where the same thresholds for generating histogram bin features were used. In other embodiments that combine phenotypic and molecular profiling, the molecular profile is determined by contacting a biological sample from said subject with reagents suitable for detecting expression levels of at least one gene product related to at least one of angiogenesis, response to hypoxia, and vasculature development, and detecting levels of expression of at least one gene product related to at least one of angiogenesis, response to hypoxia, and vasculature development, wherein a deviating level of expression of said at least one gene product related to at least one of angiogenesis, response to hypoxia, and vasculature development in comparison to a reference may indicate an increase in intra-tumor angiogenesis pathways. In some embodiments, a deviating profile that is indicative of said subject's susceptibility to anti-angiogenic therapy is characterized by an increase in intra-tumor angiogenesis pathways and elevated quantitative image features.

In certain embodiments, the molecular profile is determined by detecting the expression level of gene products from at least 1, 2, 3, 4, 5, 6, 7, 8, 9, 10, 11, 12, 13, 14, 15, 16, 17, 18, 19, 20, 30, 40, 50, 60 gene(s) selected from the group consisting of ACVRL1, AGGF1, ALAS2, AMOT, ANG, ANGPTL3, ANGPTL4, ARNT2, ATPIF1, BNIP3, BTG1, C1GALT1, CANX, CCM2, CD24, CDH13, CHRNA4, CHRNA7, CHRNB2, CLDN3, COL4A2, COL4A3, CREBBP, CULT, CXCR4, EGF, EGFL7, EGLN1, EGLN2, EMCN, EP300, EPAS1, EPGN, ERAP1, FOXC2, FOXO4, GLMN, HIF1A, HSP90B1, HTATIP2, IL17F, IL18, IL8, MYH9, MT3, NARFL, NCL, NF1, NOTCH4, NPPB, NPR1, PDIA2, PDPN, PF4, PLG, PLOD1, PLOD2, PML, PROK2, RASA1, RHOB, RNH1, ROBO4, RUNX1, SCG2, SERPINF1, SHH, SMAD3, SMAD4, SPHK1, SPINK5, STAB1, TGFB2, THY1, TNFSF12, TNNI3, VEGF-A, or subsets thereof.

In some embodiments, the gene product is a messenger RNA, while in other embodiments the gene product is a protein.

In a second aspect, the present invention provides a method for selecting a treatment for a subject suffering from a brain tumor who may be susceptible to anti-angiogenic therapy, comprising determining quantitative perfusion image features from tissue of said brain tumor to determine said subject's tumor phenotypic angiogenic perfusion profile, and comparing said subject's tumor phenotypic angiogenic perfusion profile with a reference phenotypic angiogenic perfusion profile, wherein a deviating profile for said subject relative to said reference profile identifies said subject as susceptible to anti-angiogenic therapy, and selecting for the subject, if found susceptible to anti-angiogenic therapy, an anti-angiogenic treatment to be administered in addition to chemotherapy and/or radiation therapy. Before treatment, in an additional step, said subject's tumor phenotypic angiogenic profile can be further defined with a molecular profile obtained from gene expression or protein expression analysis to create a phenotypic and molecular tumor angiogenic profile from said subject which is then compared to a reference phenotypic perfusion and molecular tumor angiogenic profile, wherein a deviating profile for said subject relative to said reference profile identifies said subject as susceptible to anti-angiogenic therapy.

In one embodiment of the present invention, the brain tumor is glioblastoma. In some embodiments, the quantitative image features are determined by measuring perfusion-weighted image data using magnetic resonance imaging that quantify regional variation and intra-tumor heterogeneity. In some embodiments, these features include, but are not limited to, mean, median, variance, maximum, skewness, kurtosis, 20 histogram bins of perfusion voxel values within the tumor region from rCBV values ranging from 0.5 to 10 at an increment of 0.5, and 20 perfusion elevated features quantifying elevated perfusion tumor burden, which is the fraction of the tumor with rCBV voxel values greater than a threshold (rCBV_{elevated}), where the same thresholds for generating histogram bin features were used. In other embodiments that combine phenotypic and molecular profiling, the molecular profile is determined by contacting a biological sample from said subject with reagents suitable for detecting expression levels of at least one gene product related to at least one of angiogenesis, response to hypoxia, and vasculature development and detecting levels of expression of at least one gene product related to at least one of angiogenesis, response to hypoxia, and vasculature development, wherein a deviating level of expression of said at least one gene product related to at least one of angiogenesis, response to hypoxia, and vasculature development in comparison to a reference may indicate an increase in intra-tumor angiogenesis pathways. In some embodiments, a deviating profile that is indicative of said subject's susceptibility to anti-angiogenic therapy is characterized by an increase in intra-tumor angiogenesis pathways and elevated quantitative image features.

In certain embodiments, the molecular profile is determined by detecting the expression level of gene products from at least 1, 2, 3, 4, 5, 6, 7, 8, 9, 10, 11, 12, 13, 14, 15, 16, 17, 18, 19, 20, 30, 40, 50, 60 gene(s) selected from the group consisting of ACVRL1, AGGF1, ALAS2, AMOT, ANG, ANGPTL3, ANGPTL4, ARNT2, ATPIF1, BNIP3, BTG1, C1GALT1, CANX, CCM2, CD24, CDH13, CHRNA4, CHRNA7, CHRNB2, CLDN3, COL4A2, COL4A3, CREBBP, CULT, CXCR4, EGF, EGFL7, EGLN1, EGLN2, EMCN, EP300, EPAS1, EPGN, ERAP1, FOXC2, FOXO4, GLMN, HIF1A, HSP90B1, HTATIP2, IL17F, IL18, IL8, MYH9, MT3, NARFL, NCL, NF1, NOTCH4, NPPB, NPR1, PDIA2, PDPN, PF4, PLG, PLOD1, PLOD2, PML, PROK2, RASA1, RHOB, RNH1, ROBO4, RUNX1, SCG2, SERPINF1, SHH, SMAD3, SMAD4, SPHK1, SPINK5, STAB1, TGFB2, THY1, TNFSF12, TNNI3, VEGF-A, or subsets thereof. In some embodiments, the gene product is a messenger RNA, while in other embodiments the gene product is a protein.

The methods of the present invention include detecting expression of at least one, two, three, four, or more genes in a biological sample from the patient. The biological sample can be, for example, tumor tissue or a blood, plasma or serum sample.

In the methods described above, the anti-angiogenic treatment can be carried out with agents that interfere with the signaling pathways of the vascular endothelium growth factor (VEGF), VEGF-receptors, angiopoietins or that are vascular disrupting agents, including, but not limited to, angiocept, bevacizumab, cilengitide, enzastaurin, sorafenib, thalidomide, thalidomide, vandetanib, nintedanib, pazopanib, cediranib, sunitinib, vatalanib, trebananib, fosbretabulin, combretastatin A4, and various combinations thereof.

The above summary is not intended to include all features and aspects of the present invention nor does it imply that the invention must include all features and aspects discussed in this summary.

### INCORPORATION BY REFERENCE

All publications, patent applications and patents mentioned in this specification are herein incorporated by reference to the same extent as if each individual publication or patent application was specifically and individually indicated to be incorporated by reference.

### BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates the procedure to generate quantitative perfusion-weighted imaging (PWI) features from perfusion images. (A) The enhancing tumor region (excluding central necrosis) was segmented on T1 images. rCBV maps were derived from perfusion images. The T1 images and the segmented tumor masks were registered to the perfusion images. Perfusion voxel values in the enhancing tumor region were extracted, which were then used to compute quantitative PWI features. (B) An illustration of computation of an imaging feature, rCBV_{elevated}_{_}_{3.5} that measures the percentage of the tumor with voxel rCBV values greater than 3.5. The red histogram bins greater than 3.5 correspond to the tumor voxels colored in red in the inset.

FIG. 2 illustrates unsupervised clustering in the cohorts from a local medical center (MC) and The Cancer Genome Atlas (TCGA). Consensus clustering of patients based on PWI features in the (A) MC and the (B) TCGA cohorts consistently identified two clusters that were well separated, as shown by the T-SNE plots of the (C) MC and the (D) TCGA cohorts. In the consensus matrices in (A) and (B), solid blue indicates the two samples always cluster together in one group, whereas white indicates they never cluster together.

FIG. 3 shows Kaplan Meier curves of patients dichotomized into two clusters. Clusters I and II in both cohorts revealed that patients in Cluster II have significantly worse survival than those in Cluster I. (A) Kaplan Meier Curve for the two clusters in the TCGA cohort (log-rank p=0.0092, HR=2.30). (B) Kaplan Meier Curve for the two clusters in the MC cohort (log-rank p=0.0041, HR=2.58). Three patients in Cluster I were removed due to missing overall survival information. (C) Box plot of patients' overall survival stratified by gene expression-based subgroup and PWI-based subtype. Right-censored patients were included in the subtype visualization, because the overall survival of each right-censored patient was above the median survival of its corresponding subtype. Here, PWI-based subtype group 2 corresponds to Cluster II, and PWI-based subtype group 1 to Cluster I.

FIG. 4 shows two clusters of GBM patients with distinct PWI image features, as illustrated by example cases of three features observed on representative image slices (the analysis was performed in 3D). Left: matrix of patients (columns) and the quantitative image features of GBM CEL regions (rows). Right: Colored perfusion maps superimposed on the aligned anatomical T1 images show example images of three linked PWI features in the two clusters with their actual values specified on the top. In the two example images for rCBV_{bin}_{_}₁, yellow indicates the percentage of voxel with values between 0.5 and 1, and purple indicates voxel values≥1 or <0.5. In the example images for rCBV_{elevated}_{_}₃ and rCBV_{elevated}_{_}₄, red represents voxels above the threshold, and those below are colored in blue. Thus, the rCBV_{elevated} feature is the proportion of the red area of the whole tumor.

FIG. 5 illustrates that anti-angiogenic treatment significantly improves overall survival of patients in Cluster II. In the subgroup of patients who were predicted to respond to anti-angiogenic treatment based on PWI features (Cluster II), patients treated with anti-angiogenic therapies are associated with significantly longer survival times than those who did not receive an anti-angiogenic therapy (log-rank p=0.022).

FIG. 6 shows histograms of all tumor PWI voxels pooled across all cases in the TCGA and MC cohorts, respectively. The histogram of pooled voxel values of the TCGA cohort (cyan) has a heavier tail than that of the MC cohort (Friedman et al., 2009). Note that the overlap between the two histograms formed the third color in the figure. This “batch effect” between the two cohorts was subsequently corrected by quantile-normalizing pooled tumor voxel values of the MC cohort based on those of the TCGA cohort. The histogram of quantile normalized voxels values of the MC cohort became identical to the histogram of the TCGA cohort (cyan).

FIG. 7 shows the identification of two clusters in the MC cohort. (A) Consensus clustering matrix results for the numbers of clusters (k ranging from 2 to 6). Both the rows and the columns are samples, where solid blue indicates that two samples always cluster together in one group, whereas white indicates two samples never cluster together. (B) Consensus cumulative distribution function (CDF) for k=2 to k=6. (C) Silhouette plot for evaluating the robustness of the discovered clusters. Each horizontal bar represents the silhouette width of a sample, and the average silhouette width of all samples in the MC cohort is 0.66. (D) Visualization of the two identified clusters in the MC cohort using MDS, consistent with FIG. 2C.

FIG. 8 shows the identification of two clusters in the TCGA cohort. (A) Consensus clustering matrix results for k=2 to 6 in the TCGA cohort. (B) Consensus CDF for k=2 to k=6. (C) Silhouette plot for evaluating the robustness of the two discovered clusters. The average silhouette width of all samples in TCGA was 0.59. (D) Visualization of the two identified clusters using MDS, consistent with FIG. 2D.

FIG. 9 shows the identification of two clusters in the MC cohort using PWI features extracted from raw tumor voxel values without quantile normalization. The two clusters are identical to those identified using quantile normalized data in FIG. 7. (A) Consensus clustering matrix results for the numbers of clusters (k ranging from 2 to 6). (B) Consensus cumulative distribution function (CDF) for k=2 to k=6. (C) Silhouette plot for evaluating the robustness of the discovered clusters. Each horizontal bar represents the silhouette width of a sample, and the average silhouette width of all samples in the MC cohort is 0.66. (D) Visualization of the two identified clusters in the MC cohort using MDS, consistent with FIG. 7D from quantile-normalized data. (E) T-SNE plot for the two clusters discovered using PWI features extracted from raw tumor voxel values.

FIG. 10 illustrates the intra- and inter-tumor heterogeneity in tumor perfusion MR images. Perfusion rCBV color maps in CEL tumor regions superimposed onto grey-scale T1-weighted images show regional variation in perfusion within tumors and across tumors. rCBV values were discretized into 20 bin ranging from 0.5 to 10, where red color indicates high rCBV values and blue color indicates low rCBV values.

FIG. 11 shows full color maps of the perfusion rCBV images in FIG. 4. rCBV maps in the tumor regions were superimposed on T1-weighted images.

FIG. 12 shows two example cases showing that lower rCBV_{elevated}_{_}_{3.5} was associated with better survival (top, overall survival (OS): 1228 days), and higher rCBV_{elevated}_{_}_{3.5} was associated worse survival (bottom, OS: 123 days). From left to right, the original T1-weighted image with ROI drawn around the tumor (left 1), the perfusion rCBV map (left 2), the color map of the tumor at a threshold of 3.5, where red are voxels greater than 3.5 and blue are voxels less than 3.5, and histogram to generate the value of the feature (right).

FIG. 13 illustrates PWI features ranked by gini index in random forest models in the two cohorts, with recursive best subsets of features colored in red. (A) The best subset PWI features found by recursive feature selection in the TCGA cohort are colored in red. (B) The best subset PWI features in the MC cohort are colored in red.

FIG. 14 shows correlation matrices of PWI features for the two cohorts. (A) The correlation matrix of the PWI features in the MC cohort showing that many features are highly correlated. (B) Highly correlated features are similarly observed in the correlation matrix of the PWI features in the TCGA cohort.

FIG. 15 shows flowcharts of anti-angiogenic information available in the two cohorts studied herein. *One case was removed due to unavailability of overall survival.

### DETAILED DESCRIPTION

Before describing specific embodiments of the invention, it will be useful to set forth definitions that are utilized in describing the present invention.

### I. DEFINITIONS

The practice of the present invention may employ conventional techniques of magnetic resonance imaging which are within the capabilities of a person of ordinary skill in the art. Such techniques are fully explained in the literature. For definitions, terms of art and standard methods know in the art, see, for example, Paul Tofts “Quantitative Mill of the brain: measuring changes cause by disease,” John Wiley & Sohns, 1 st edition (2003) which is herein incorporated by reference. Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used in this specification and in the appended claims, the singular forms “a” and “the” include plural referents, unless the context clearly dictates otherwise.

The term “about”, as used herein, particularly in reference to a given quantity, is meant to encompass deviations of plus or minus five percent.

The term “glioblastoma,” as used herein, refers to Glioblastoma Multiforme (GBM). GBM is the most common and most aggressive type of primary brain tumor in humans. The treatment options for GBM include radiosurgery, radiation, chemotherapy, anti-angiogenic treatment, and treatment with corticosteroids.

The terms “subject” or “patient” are used interchangeably herein and relate to a mammalian, particularly to a human being. The subject or patient may already be diagnosed with glioblastoma multiforme or may only be suspected to suffer from glioblastoma multiforme.

The term “control subject,” as used herein, may refer to a subject who was diagnosed with glioblastoma multiforme but whose molecular subtype of glioblastoma multiforme is deemed not to responsive to anti-angiogenic treatment.

Anti-angiogenesis or anti-angiogenic treatment is directed to arrest and shut down the formation of new blood vessels that grow in response to angiogenic factors that solid tumors including glioblastoma produce to allow tumor expansion, progression, and eventually tumor metastasis. Anti-angiogenic treatment, generally as an addition to standard chemotherapy, radiation or radiosurgery, can be efficacious in difficult-to-treat cancers including glioblastoma, but only if the glioblastoma patient is susceptible to the anti-angiogenic treatment. Anti-angiogenic agents, in most cases, interfere with the signaling pathways of the vascular endothelium growth factor (VEGF) and VEGF-receptors and are, in most cases, small molecules or (humanized) monoclonal antibodies including, but not limited to, angiocept, bevacizumab, cilengitide, enzastaurin, sorafenib, thalidomide, thalidomide, vandetanib, nintedanib, pazopanib, cediranib, sunitinib, vatalanib. Newer developments also include signaling pathway inhibitors of angiopoietins (vascular growth factors) and vascular disrupting agents (VDAs) which specifically target newly formed blood vessels within the tumor, and various combinations of anti-angiogenic agents (Monk et al., 2016; Mita et al., 2013). Angiopoetin-targeting anti-angiogenic therapy includes agents such as trebananib, while VDAs include agents such as fosbretabulin and its active metabolite combretastatin A4 (Monk et al., 2016).

VEGF and VEGF-A refer to the full-length as well as truncated parts of the human as well as non-human vascular endothelial cell growth factor and are part of the VEGF family including VEGF-B, VEGF-C, VEGF-D, VEGF-E, VEGF-F, and PIGF.

The nuclear factor kappaB family and cascade of transcription factors is involved in a wide range of biological processes including, but not limited to, innate and adaptive immunity, inflammation, B-cell development, lymphoid organ formation, stress responses, cell survival, cell proliferation, and more. The cascade is rapidly set into motion in response to stimulation by proinflammatory and immunomodulatory cytokines (e.g. TNF, IL-1, IL-2, IL-6), chemokines, leukocyte adhesion molecules, anti-apoptotic genes, immune cells, and facilitates the expression of target genes required in such biological processes (Solt & May, 2008). In cases of chronic inflammatory disorders and certain types of tumors, the response to such stimulation becomes dysregulated.

The endoplasmatic reticulum (ER) has a key function in the production, glycosylation, folding and sorting of secreted proteins which requires a properly balanced oxidative environment with oxidases, peroxidases and folding catalysts. An imbalance in the oxidative environment can lead to the accumulation of unfolded proteins causing ER stress and can affect angiogenesis via the pathway of the unfolded protein response.

The term “voxel,” as used herein, denotes a volume element that corresponds to a discrete image element (pixel) and is used to express a quantity in a unit per volume of tissue.

The term “non-invasive,” as used herein, refers to methods for obtaining data for assessment without the need for an invasive surgical intervention or invasive medical procedure.

The terms “diagnostic” and “diagnosis,” as used herein, refer to the determination of a molecular subtype of glioblastoma multiforme that is responsive to anti-angiogenic treatment, and can comprise the determination of the presence of glioblastoma, the monitoring of the course of glioblastoma, the staging of glioblastoma, and the monitoring of a glioblastoma patient's response to therapeutic intervention, particularly to anti-angiogenic treatment.

The term “gene set enrichment analysis,” as used herein, refers to a method to identify up-regulated gene sets and molecular pathway activities within clusters that are established based on quantitative PWI features.

Magnetic resonance imaging (MRI) allows to noninvasively image body tissues such as the brain based on the electromagnetic activity of atomic nuclei. Nuclei consist of protons and neutrons, both of which have spins and can induce their own magnetic field through their motion. Clinically, hydrogen nuclei (water protons) are most often used because of their abundance in the body and because they are the most convenient molecular species to study.

MRI is carried out by exciting protons in a uniform magnetic field out of their low-energy equilibrium state through a radiofrequency (RF) pulse and measuring electromagnetic radiation that is released while the protons decay back to the low-energy equilibrium level. In an MRI scanner a radiofrequency transmitter is used to produce an electromagnetic field, whereby the strength of the magnetic field is influenced by the intensity and the duration of the radiofrequency. When the body is subjected to a magnetic field within an MRI scanning machine, some protons get excited, their electromagnetic moments change and align with the direction of the external magnetic field, i.e., their spin direction gets flipped. Once the external magnetic field is turned off, the excited protons decay to their original equilibrium spin state, thereby releasing the differential energy as photons. It is these photons that produce the electromagnetic signal that the MRI scanning machine ultimately detects (MR signal). Since the protons in different tissues return to their equilibrium state at different rates, an image can be constructed. In the course of this process, MRI scanners generate multiple two-dimensional cross sections or slices of tissue and reconstruct 2- or 3-dimensional imagines that can provide valuable information about the local tissue environment and potentially provide diagnostic indication of pathological conditions in a particular region of interest (ROI).

An MRI system typically consists of several components: a) a magnet to produce a magnetic field; b) coils to make the magnetic field homogenous; c) a radiofrequency transmitter (radiofrequency coil) to transmit a radio signal into the body part or tissue being imaged; d) a receiver coil to detect the returning radio signals; e) gradient coils to provide spatial localization of the radio signals; f) a computer-readable medium or computer to reconstruct the radio signals into an MRI image using specific algorithms and to subject to further analysis.

Quantile normalization, a multi-sample normalization technique, was used herein to correct the experimental data high-throughput data for technical variability.

By identifying a region within a subject's brain that is unaffected by glioblastoma and with a relatively constant physiological state for the intended duration of anti-cancer treatment and, optionally, treatment monitoring, the signal intensity of this region in the subject's brain can be used to normalize the image data set. By normalizing volumetric regions, such as the cerebral blood volume, to the white matter in the subject's brain, the relative cerebral blood volume is determined.

Registration is used herein to align images to detect changes that provide insight into the progression of glioblastoma. The images can be obtained from various imaging modalities, for example, but not limited to magnetic resonance imaging (MRI), computed tomography (CT), two-dimensional planar X-Ray, positron emission tomography (PET), ultrasound (US), optical imaging (i.e. fluorescence, near-infrared (NIR) & bioluminescence), and single-photon emission computed tomography (SPECT).

Within a given instrumentation source including, but not limited to, MRI, CT, X-Ray, PET, SPECT, data can be generated by diffusion, perfusion, permeability, normalized and spectroscopic images, which include molecules containing, for example, 1H, 13C, 23-Na, 31P, and 19F.

The techniques of the present disclosure are not limited to a particular type of tissue region and are generally useful for all soft tissues. The tissue may be soft tissue such as brain, and may be tumorous and indicative of a benign or malignant brain tumor, or non-tumorous.

### II. WAYS OF MAKING AND USING THE INVENTION

The present invention is based on the inventors' discovery that quantitative perfusion-weighted magnetic resonance imaging, optionally combined with intra-tumor specific molecular profiling, can be used to predict treatment response of glioblastoma multiforme (GBM) patient subtypes to anti-angiogenic therapy. Patient subtypes with high intratumor quantitative perfusion-weighted imaging (PWI) features had elevated levels of hypoxia pathways and angiogenesis, and were found to be susceptible to anti-angiogenic treatment. Upon anti-angiogenic treatment, those patient subtypes with high intra-tumor PWI features experienced a higher survival rate than patient subtypes who lacked the intra-tumor PWI features. Since GBM has a very poor survival rate due to the lack of effective treatments and since only a fraction of GBM patients is susceptible to anti-angiogenic treatment, it is very important to have a reliable methodology available to identify this fraction of GBM patients so that a targeted, survival-prolonging anti-angiogenic treatment approach can be initiated as soon as possible. In order to further an understanding of the invention, a more detailed discussion is provided below regarding computer-based methods to noninvasively identify subtypes of glioblastoma multiforme (GBM) patients who are susceptible to anti-angiogenic treatment based on their quantitative perfusion-weighted imaging features and molecular profile.

### Brain Tumors

Such methods, as described herein, are applicable to all astrocytomas, in particular to glioblastoma, but can also be advantageous in treating other malignant brain tumors, e.g. medulloblastoma, neuroglioma, oligodendroglioma, meningioma, ependymoma, etc.

Glioblastoma multiforme (GBM) is the most commonly occurring, malignant and fast-growing astrocytoma in adults, particularly between the ages of 45 to 70 years old, and accounts for about 15 percent of all brain tumors. Particular characteristics of GBM are focal necrosis and endothelial proliferation, which in turn can induce angiogenic activity. Since general chemotherapy and radiation therapy fail to provide a long-term effect for GBM, most affected patients die within 15 months of diagnosis.

Identification of Distinct Glioblastoma Multiforme (GBM) Molecular Subtypes

Studies of gene expression of the brain provide insights into the different physiological and pathological states of the brain. Differential gene expression studies allow to identify molecular subtypes of tumors based on intertumor molecular heterogeneity as well as intratumor molecular heterogeneity, which may predict the various clinical responses upon anti-tumor treatment (Tarca et al., 2006; Phillips et al., 2006). Transcripts indicative of differential gene expression can be identified through a variety of methods known to those skilled in the art, including, but not limited, to microarray expression profiling, differential screening, differential display, competition hybridization, substractive hybridization, expressed sequence tag sequencing of cDNA libraries, serial analysis of gene expression (SAGE).

An inquiry led by the TCGA into the molecular characteristics of GBM found that GBM is not a uniform disease, but that GBM manifests itself in various distinct molecular subtypes where patients within one subtype respond to chemotherapy and radiation therapy differently than patients within another subtype (TCGA Research Network, 2008).

Based on their gene expression pattern, molecular subtypes were designated as classical, non-G-CIMP, G-CIMP, mesenchymal, proliferative, neural, and proneural (Phillips et al., 2006.

### Gene Set Analysis

Gene set analysis was performed to identify sets of genes that are functionally related or jointly or cumulatively associated with angiogenesis, hypoxia pathways, vasculature development, and other conditions.

In particular, 13 gene sets were evaluated for differential expression between patient subtypes, as described below in Example Three and Table 2, including: 1) Nuclear Factor(NF)-KappaB cascade and 1-KappaB Kinase/NF-KappaB cascade; 2) cytokine activity, 3) response to hypoxia, 4) regulation of 1-KappaB Kinase/NF-KappaB cascade, 5) anatomical structure formation, 6) hydrolase activity hydrolyzing 0-glycosyl compounds, 7) angiogenesis, 8) oxidoreductase activity, 9) vasculature development, 10) positive regulation of 1-KappaB Kinase/NF-KappaB cascade, 11) Endoplasmic reticulum (ER) Golgi intermediate compartment, 12) oxidoreductase activity acting on the CH—CH group of donors, and 13) response to wounding.

As also described in Example Three, subsequent gene set enrichment analysis (GSEA) showed that the glioblastoma subtype that was identified with methods of the present invention as being susceptible to anti-angiogenic treatment was particularly enriched for genes in the gene sets for the response to hypoxia, angiogenesis, and vasculature development.

### Response to Hypoxia

Response to Hypoxia denotes a change in state or activity of a cell or an organism in terms of movement, secretion, enzyme production, gene expression, etc. as a result of a stimulus indicating lowered oxygen tension. Oxygen is a key substrate in cellular metabolism and the main reason for neovascularization in tumors. In a pathological state, like it is the case with tumor growth, oxygen is often not available in sufficient amounts. Cells of aerobic organisms that experience hypoxic (oxygen-deprived) conditions temporarily halt cell division to reduce their energy consumption and start to secrete proangiogenic factors, involving pathways such as mTOR signaling, unfolded protein response, hypoxia inducible factors (HIFs), to facilitate neovascularization and survival.

Genes related to the response to hypoxia that were part of the gene set tested included ALAS2, ANG, ARNT2, BNIP3, CD24, CHRNA4, CHRNA7, CHRNB2, CLDN3, CREBBP, CXCR4, EGLN1, EGLN2, EP300, EPAS1, HIF1A, HSP90B1, MT3, NARFL, NF1, PDIA2, PLOD1, PLOD2, PML, SMAD3, SMAD4, TGFB2, VEGF-A.

From this gene set, an upregulation in the susceptible glioblastoma subtype of the following genes war particularly noticeable: VEGF-A, PLOD2, PLOD1, HSP90B1, ANG, EGLN1, BNIP3, EPAS1, TGFB2, CXCR4.

### Angiogenesis

Angiogenesis, the formation of new blood vessels from the proliferation of pre-existing blood vessels, is instrumental in many physiologic and pathologic processes involving endothelial cells and extracellular matrix, and is modulated by signaling pathways, cell-matrix interactions, matrix remodeling enzymes, growth factors including, but not limited to, vascular endothelial growth factor (VEGF), fibroblast growth factor (FGF), tumor necrosis factor-alpha (TNF-alpha), transforming growth factor-beta (TGF-beta), angiopoietins, and more (Ucuzian et al., 2010).

Endothelial cells have the capacity to form lumens within preexisting vasculature to allow for the development of new capillary networks. Although highly prevalent in tumorigenesis, angiogenesis also occurs in wound healing, where it contributes to the adaptive repair response.

Genes related to angiogenesis that were part of the gene set tested included ACVRL1, AGGF1, AMOT, ANG, ANGPTL3, ANGPTL4, ATPIF1, BTG1, C1GALT1, CANX, CDH13, CHRNA7, COL4A2, COL4A3, EGF, EMCN, EPGN, ERAP1, FOXO4, HTATIP2, IL17F, IL18, IL8, MYH9, NCL, NF1, NOTCH4, NPPB, NPR1, PF4, PLG, PML, PROK2, RHOB, RNH1, ROBO4, RUNX1, SCG2. SERPINF1, SHH, SPHK1, SPINK5, STAB1, TGFB2, THY1, TNFSF12, TNNI3, VEGF-A.

From this gene set, an upregulation in the susceptible glioblastoma subtype of the following genes war particularly noticeable: VEGF-A, IL8, RNH1, CANX, ANG, ANGPTL4, COL4A2, MYH9, RUNX1, PF4, EGF, TGFB2, NPPB, AGGF1, NOTCH4.

### Vasculature Development

Vasculature development refers to the process whose specific outcome is the progression of the vasculature over time, from its formation to the mature structure.

Genes related to vasculature development that were part of the gene set tested included ACVRL1, AGGF1, AMOT, ANG, ANGPTL3, ANGPTL4, ATPIF1, BTG1, C1GALT1, CANX, CCM2, CDH13, CHRNA7, COL4A2, COL4A3, CUL7, EGF, EGFL7, EMCN, EPGN, ERAP1, FOXC2, FOXO4, GLMN, HTATIP2, IL17F, IL18, IL8, MYH9, NCL, NF1, NOTCH4, NPPB, NPR1, PDPN, PF4, PLG, PML, PROK2, RASA1, RHOB, RNH1, ROBO4, RUNX1, SCG2, SERPINF1, SHH, SPHK1, SPINK5, STAB1, TGFB2, THY1, TNFSF12, TNNI3, VEGF-A.

From this gene set, an upregulation of the following genes war particularly noticeable in the susceptible glioblastoma subtype: VEGF-A, IL8, RNH1, CANX, ANG, ANGPTL4, COL4A2, PDPN, MYH9, RUNX1, PF4, EGF, CUL7, TGFB2, NPPB, AGGF1, NOTCH4.

### Discrimination Between Patient Subtypes Based on Molecular Profiling

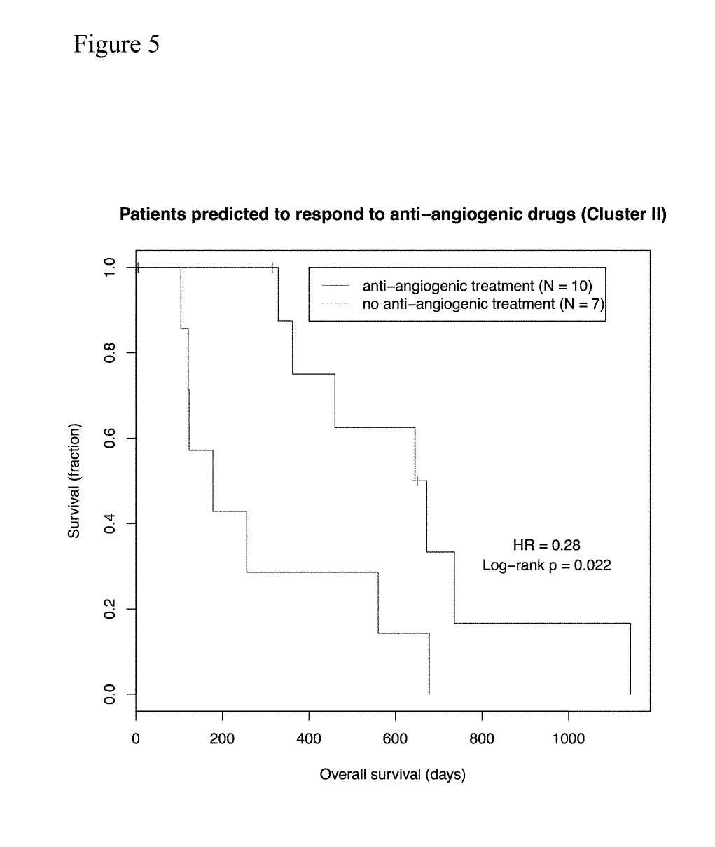

In order to be able to discriminate between two or more patient subtypes, e.g. patient subtypes who are susceptible or not susceptible to anti-angiogenic treatment, for a defined set of molecular profiles, the inventors of the present invention applied a machine learning approach including, but not limited to, hierarchical clustering and random forest classifying. This approach led to an algorithm that was trained by reference data, thus by data of reference molecular profiles defining the two or more patient subtypes, e.g. susceptible or not susceptible to anti-angiogenic treatment, for the defined set of molecular profiles to discriminate between the two or more patient subtypes. The inventors found that this approach yielded two glioblastoma subtype clusters with distinct perfusion-weighted imaging features where one cluster (here cluster II) was correctly predicted to be susceptible to anti-angiogenic treatment, as illustrated in FIG. 5.

An exemplary approach to discriminate between patient subtypes that are or are not predicted to be susceptible to anti-angiogenic treatment is summarized as follows:

Step 1: Regions of interest are manually drawn using axial T1-weighted images, and volumetric contrast-enhancing lesion (CEL) regions are deduced from the difference between the image voxels contained within the entire tumor and those contained within the region of central necrosis. The T1 and the CEL ROI volumes are then registered to the perfusion MR volume.

Step 2: The perfusion-weighted images are created using T2*-weighted gradient-echo echo planar imaging. Quantitative voxel-based perfusion-weighted imaging (PWI) features are generated from the enhancing regions of the GBM tumors. Relative cerebral blood volume (rCBV) maps are generated using perfusion analysis, and the perfusion values generated are normalized to the normal-appearing white matter in the hemisphere contralateral to that of the GBM tumor.

Step 3: The volumes of the transformed tumor ROI and the rCBV map are superimposed to extract voxel-based rCBV values in the enhancing region of the GBM tumor. This registration step consists of: 1) skull stripping to remove the skull from the T1-weighted imaging volume, 2) initializing the registration by aligning the center of the head in the T1- and PWI-weighted image volumes. 3) Establishing an affine linear transformation to map the T1-weighted to the PWI-weighted image volume, and 4) applying the affine transform to the tumor ROI volume. After this registration step, the transformed tumor ROI is aligned with the rCBV map in the same coordinate space, and rCBV voxel values in the enhancing ROI are extracted.

Step 4: The rCBV voxel values in the enhancing region of the GBM tumor are used to quantify features that capture perfusion image phenotypes both of the whole tumor and of tumor heterogeneity. A total of 46 non-parametric voxel-based PWI features in the CEL of each GBM tumor were quantified, including 6 summary statistics describing the bulk tumor characteristics and 40 histogram-based features quantifying regional variation and intra-tumor heterogeneity of PWI voxel values. The 6 summary statistics included mean, median, variance, maximum, skewness, and kurtosis. The histogram-based features consisted of 20 histogram bins (rCBV_{bin}) at an interval of 0.5 ranging from 0.5 to 10, and 20 features that measure elevated perfusion tumor burden—the fraction of the tumor with rCBV voxel values greater than a threshold (rCBV_{elevated}), where the same thresholds were used for generating histogram bins.

Determining Functional Phenotypes from Dynamic Susceptibility-Weighted Contrast-Enhanced Perfusion Images

Perfusion-weighted imaging (PWI) of the brain provides insights into the extent and speed with which blood reaches the various portions within the brain. Due to pathological tissue changes and possible neovascularization due to tumor angiogenesis, tumorous brain tissue exhibits an altered perfusion and vascular permeability.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### III. EXAMPLES

### Experimental Procedures

The following methods and materials were used in the examples that are described further below.

### Patient Cohorts

HIPPA-compliant institutional review board approval was obtained with informed consent for all patients. Patients 18 years of age or older with de novo GBM who underwent three-dimensional pre-surgical gadolinium-based contrast-enhanced T1-weighted and DSC T2*-weighted perfusion MR imaging exams were retrospectively acquired from two independent patients cohorts.

The first cohort consisted of 68 patients in the Cancer Imaging Archive (TCIA) collected from two institutions. Patient-matched microarray gene expression data, gene expression-based subtypes previously defined by The Cancer Genome Atlas (TCGA), clinical chemotherapy drug information, and overall survival were downloaded from TCGA (Brennan et al., 2013). A total of 20 cases were removed from the TCGA cohort due to several data quality issues including 14 cases missing baseline pre-surgical images, 4 cases with low signaling to noise ratio (SNR) on perfusion MR images, 1 case with section thicknesses of T1 images greater than 5 mm, and 1 case with incomplete imaging series.

The second cohort comprised 79 patients from a local Medical Center (MC). A total of 10 patients were excluded from the MC cohort: 4 cases with incomplete series and 6 cases with no survival data. Thus, there were 48 patients in the TCGA cohort and 69 patients in the MC cohort used in subsequent analyses.

Anti-angiogenic chemotherapy as part of the therapeutic regimen—regardless of being adjuvant or in progression—was annotated for both cohorts. Chemotherapy information was available for 25 and 30 patients in the TCGA and MC cohorts, respectively. For the TCGA cohort, anti-angiogenic treatments included angiocept, bevacizumab (Avastin®), cilengitide, enzastaurin, sorafenib, thalidomide, thalidomide, vandetanib (Lu-Emerson et al., 2015). Among the 9 patients given anti-angiogenic therapies in the TCGA cohort, 3 were treated both in the initial treatment and at tumor progression, and the other 6 at progression or recurrence. In contrast, except for 1 patient treated with enzastaurin, Avastin® was the only anti-angiogenic therapy given to patients in the MC cohort. Among the 27 patients whose anti-angiogenic treatment dates were available in the MC cohort, 2 patients were administered adjuvant anti-angiogenic treatment concurrent with temozolomide (TMZ) as the first line therapy, and 25 received Avastin® at tumor recurrence.

### DSC MR PWI Data Acquisition Protocol

The image data of the TCGA cohort were collected from two institutions and downloaded from the Cancer Imaging Archive (Clark et al., 2013). The perfusion-weighted images from both institutions in TCGA were obtained with T2*-weighted gradient-echo echo planar imaging. The perfusion images from institution 1 (N=35) were acquired with a 1.5-T or 3-T MR machine (TE: 40 ms; TR: 1550 ms or 1900 ms; flip angle, 90°), with a section thickness of 5 or 6 mm. The perfusion images from institution 2 (N=13) were collected with a 1.5-T MR machine (TE: 54 ms; TR: 1250 ms or 2000 ms; flip angle, 30°) with section thicknesses ranging from 3, 4, to 5 mm. Perfusion images were acquired during passage of 0.1 mmol/kg gadopentetate dimeglumine (Magnevist; Bayer healthcare, Berlin, Germany) administered at a rate of 5 mL/sec for patients in both institutions in TCGA (Jain et al., 2013). Contrast bolus preload was not employed.

The T2*-weighted gradient-echo EPI perfusion images in the MC cohort (N=69) were acquired with a 1.5-T MR machine (TE: 40 ms; TR: 1800 ms or 1113 ms; flip angle, 60° or 90°) with a section thickness of 5 mm during passage of 0.1 mmol/kg of gadopentetate dimeglumine (Magnevist; Bayer healthcare, Berlin, Germany) or gadobenate dimeglumine (MultiHance, Bracco, Milan, Italy) administered at a rate of 4 mL/sec. Acquisition time was 2 minutes. Contrast bolus preload was not employed.

### Image Processing Pipeline for Computation of PWI Features

Regions of interest (ROIs) were manually circumscribed by a neurosurgery resident and a neurosurgeon by consensus to segment the entire tumor and the region(s) of central necrosis on each axial slice of the T1-weighted images, and they were subsequently reviewed by a board certified neuroradiologist (L.A.M). The ROIs were created using the OsiriX software package (OsiriX Viewer). The volumetric contrast-enhancing lesion (CEL) region was deduced by taking the difference in the image voxels contained within the entire tumor and those contained within the region of central necrosis. The T1 and the CEL ROI volumes were then registered to the perfusion MR volume automatically using a mutual information algorithm with a 12-degrees of freedom transformation in 3D Slicer (Fedorov et al., 2012; Johnson et al., 2007).

The voxel-by-voxel rCBV values were computed by integrating the area under the ΔR2* curve (Boxerman et al., 2006). The underlying algorithm for computing rCBV was optimized to improve accuracy by correcting for two opposing effects: (1) T1-weighted leakage that was likely to underestimate rCBV, and (2) T2/T2*-weighted imaging residual effect that tends to over-estimate rCBV (Hu et al., 2010; Paulson et al., 2008).

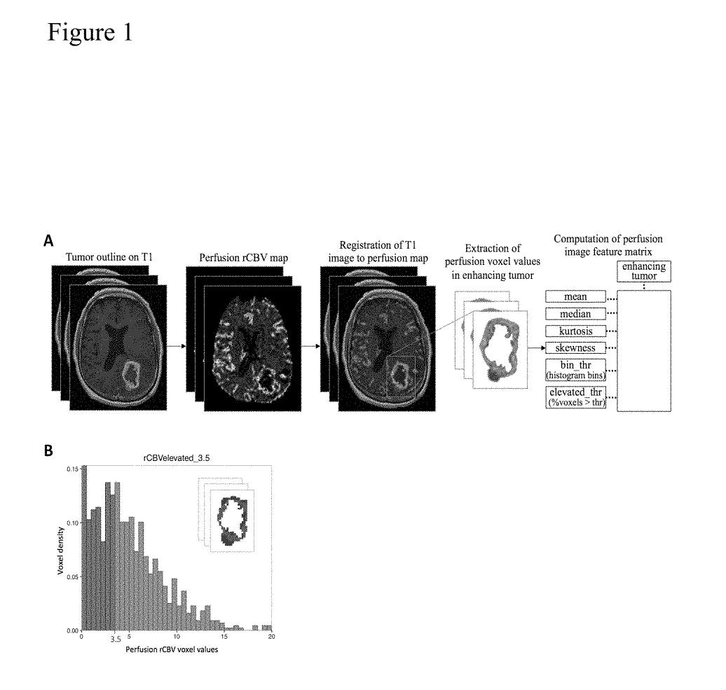

As shown in FIG. 1, an image analysis pipeline was developed and applied to generate quantitative voxel-based PWI features from the enhancing regions of the GBM tumors, similar to that previously described (Liu et al., 2016). Relative cerebral blood volume (rCBV) maps were generated using FDA-approved D3 Neuro perfusion analysis software (v1.1; Imaging Biometrics, LLC, Elm Grove, Wis., USA), a plugin integrated in the OsiriX platform. The perfusion values generated by D3 Neuro were normalized to the normal-appearing white matter (NAWM) in the hemisphere contralateral to that of the tumor. The volumes of the transformed tumor ROI and the rCBV map were superimposed to extract voxel-based rCBV values in the enhancing region of the GBM tumor, implemented in a script in Matlab (a mathworks product).

### Quantile Normalization of Pooled PWI Tumor Voxel Values Between Two Cohorts

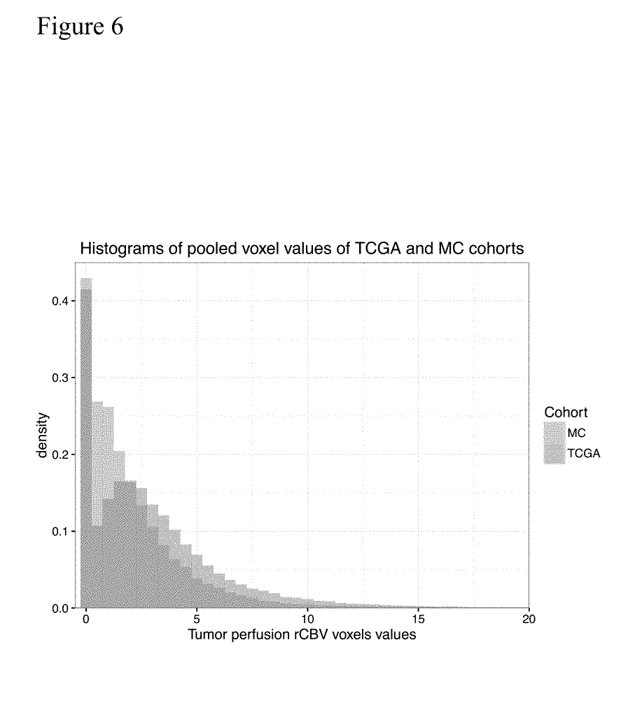

As illustrated in FIG. 6, due to variation arising from different scanners/vendors and different institutions in imaging data acquisition, there may have been “batch effects” in perfusion voxel values between the two cohorts. Batch effects are also commonly observed in molecular data, such as multiple batches of microarray experiments. Quantile normalization was widely used to correct for batch effects in molecular data (Bolstad et al., 2003). In consistency with this practice, the PWI tumor voxel values pooled from all patients between the two cohorts were quantile normalized. The voxel values of the TCGA cohort were used to quantile normalize those of the MC cohort, using the normalize.quantile.use.target function in the “preprocessCore” bioconductor R package (Bolstad et al., 2003).

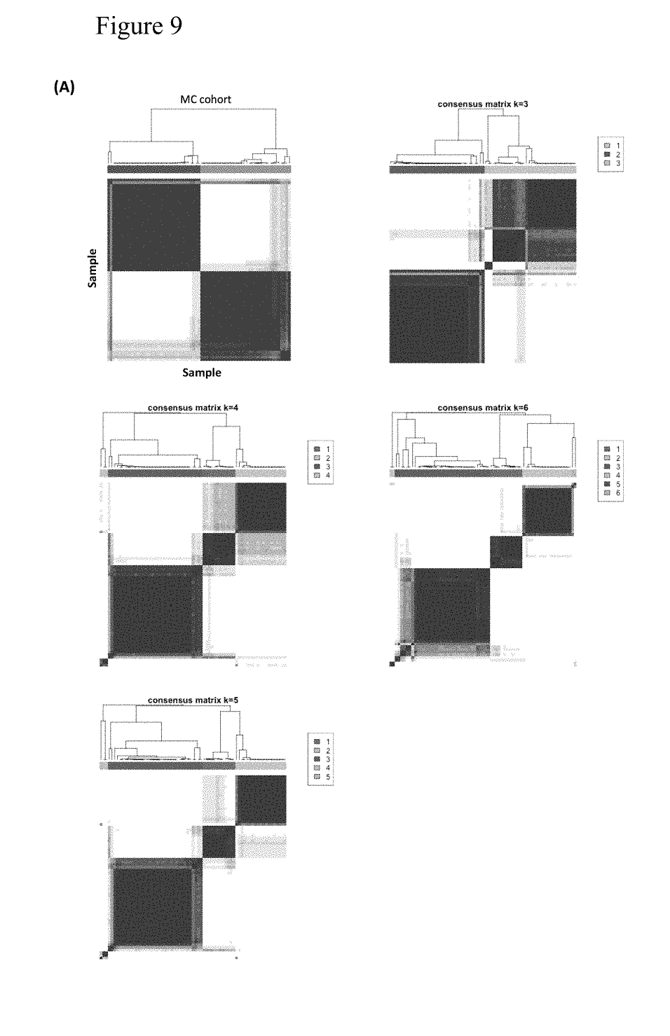

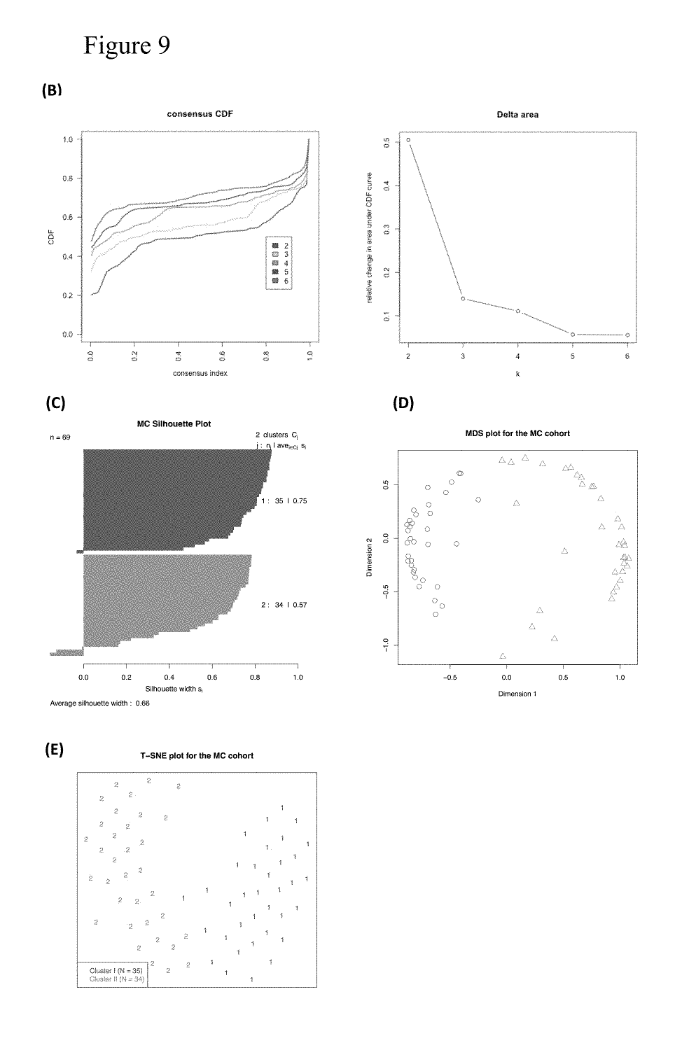

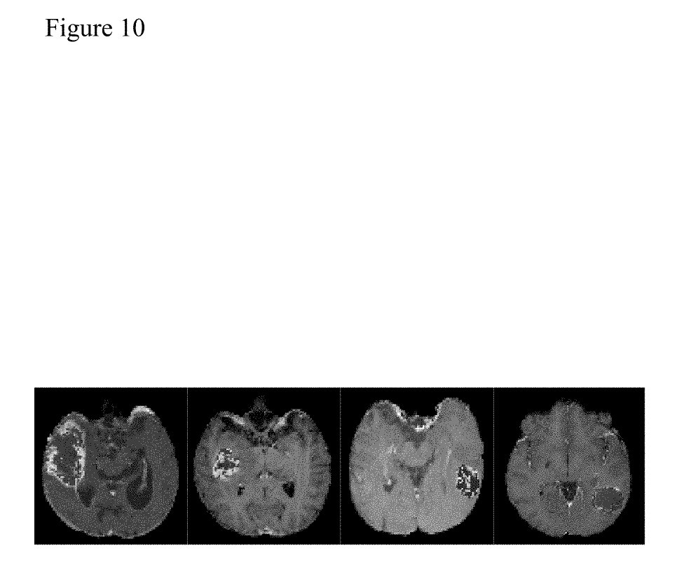

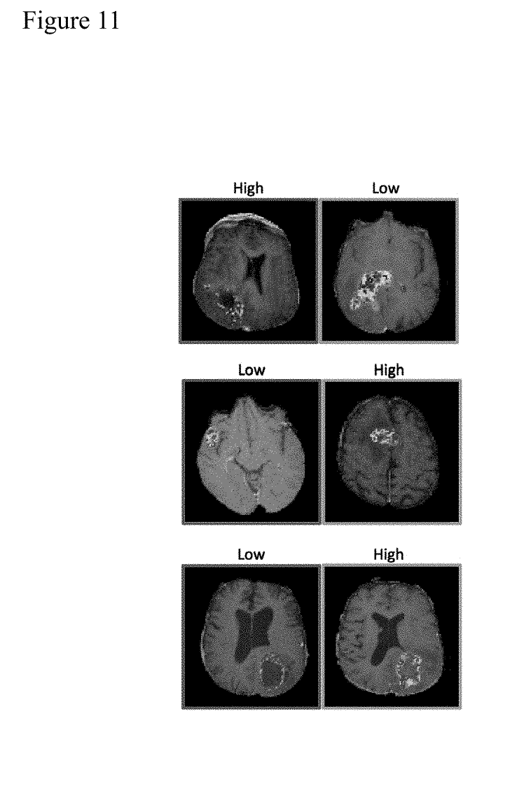

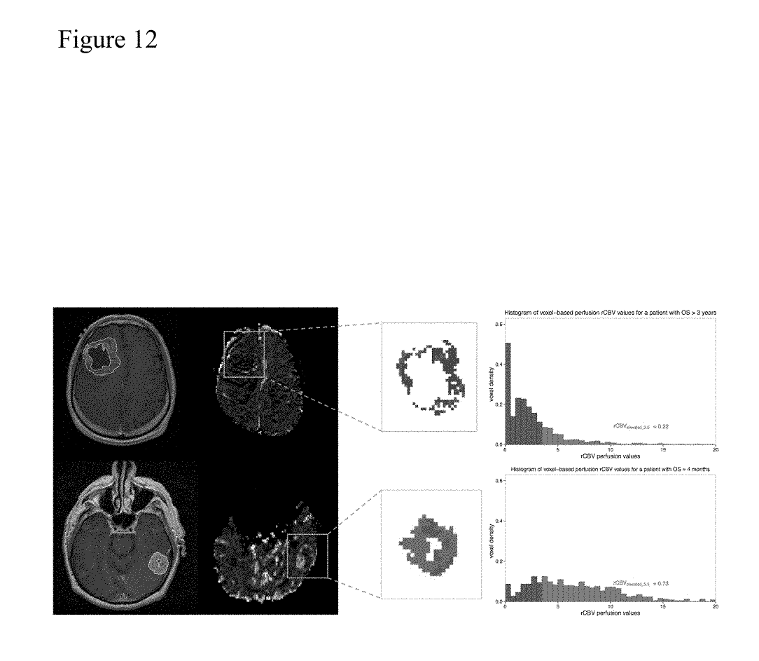

The unsupervised consensus clusters remained the same using PWI features extracted from raw tumor voxel values, invariant to the quantile normalization pre-processing step (FIG. 9). A random forest classifier was trained using the raw features of the MC cohort without quantile normalization to predict the two subgroups in TCGA, and a classifier after swapping the training and test cohorts. 79% (38 of 48) of TCGA patients and 81% (56/69) of the MC cohort predicted by the two classifiers were assigned to the same clusters as those by the unsupervised consensus clustering approach, respectively. After quantile normalization, the prediction accuracies improved to 96% in TCGA and 93% in the MC cohort.

### Quantification of PWI Features

Features that capture perfusion image phenotypes both of the whole tumor and of tumor heterogeneity were extracted. After the quantitative image analysis pipeline, a total of 46 non-parametric voxel-based PWI features in the CEL of each GBM tumor were quantified, including 6 summary statistics describing the bulk tumor characteristics and 40 histogram-based features quantifying regional variation and intra-tumor heterogeneity of PWI voxel values, as shown in FIG. 1A. The 6 summary statistics included mean, median, variance, maximum, skewness, and kurtosis (Davnall et al., 2012).

Skewness measures the symmetry of a distribution, where positive skewness has the mass of the distribution concentrated on the right (Davnall et al., 2012). Kurtosis measures the spread or peakiness of a distribution (Davnall et al., 2012).

The histogram-based features consisted of 20 histogram bins (rCBV_{bin}) at an interval of 0.5 ranging from 0.5 to 10, and 20 features that measure elevated perfusion tumor burden—the fraction of the tumor with rCBV voxel values greater than a threshold (rCBV_{elevated}), where the same thresholds were used for generating histogram bins, as shown in FIG. 1B.

### Discovery of PWI-Based Subtypes

Hierarchical consensus clustering was performed with agglomerative average linkage to discover PWI-based clusters in GBM patients (Monti et al., 2003). The PWI features were normalized by mean-centering each feature. The resulting clusters were represented and visualized using t-distributed stochastic neighbor embedding (T-SNE) implemented in R, with a pairwise distance metric of (l-r), where r is the Pearson's correlation coefficient (Maaten et al., 2008; Verhaak et al., 2010). For each possible number of clusters from 2 to 6, the algorithm was iterated 1000 times at an 80% subsampling rates of features and samples, which aggregated to a consensus matrix showing the likelihood that two samples belong to the same cluster. The maximum number of iterations was set to 2000 to keep the cost (error) below 0.5. In the training MC cohort, the optimal number of clusters was selected on the basis of the largest overall average silhouette score from k=2 to 6 that is closest to 1 (Rousseeuw, 1987).

### Multi-Dimensional Scaling (MDS)

We used multi-dimensional scaling to create a two-dimensional representation of the two discovered clusters, where the pairwise distance function was consistently defined as 1 minus the Pearson's correlation coefficient that was also used in consensus clustering analysis to generate the two clusters (Cox & Cox, 2000).

Identification of Important PWI Features Associated with Each Cluster

To validate the reproducibility of patient clusters, a random forest model (Liaw & Wiener, 2002) was built using the TCGA cohort to predict cluster assignment of the MC cohort, which was compared to the clusters identified from unsupervised consensus clustering above. Similarly, the cluster assignment of the TCGA cohort was predicted using the MC cohort, and the prediction accuracy was reported. The importance of the PWI features was evaluated using the gini index (Liaw & Wiener, 2002). Feature selection of a subset of PWI features that achieved the highest 10-fold cross validation accuracy was identified using a recursive feature elimination (RFE) algorithm implemented in an R package caret (Caret, 2008).

### Survival Analysis

The survival analysis, in general the analysis of the time between the first diagnosis of GBM and death, was either based upon one factor under investigation (univariate analysis) or upon various factors or covariates (multivariate analysis). Covariates include, but are not limited to, patient's age, tumor phenotype, gene expression-based subtype, gender, histology and so forth (Bradburn et al., 2003).

Kaplan-Meier survival analysis was performed with the log-rank test on categorical clinical variables, including age>60, gender, solitary or multi-centric tumor phenotype, gene expression-based subtypes, and the discovered PWI-based groups. These variables were also used to construct a multivariate Cox proportional hazards survival regression model to assess the clinical significance of PWI-based groups in associating with overall survival, after accounting for other clinical prognostic covariates (Cox, 1972).

The univariate Cox analysis using the expression-based subtypes showed that the non-G-CIMP Proneural subtype was significantly associated with poor survival (log-rank p=0.0053, HR=4.6), whereas no such significant association with survival was observed in the other subtypes (Table 4). In the multivariate Cox models, the PWI-based subgroup and the non-G-CIMP Proneural subtype remained significantly associated with poor survival (Tables 4 and 5).

The Kaplan-Meier survival analysis was carried out to assess the prognostic value of anti-angiogenic treatment in Cluster II patients, who were predicted to respond to anti-angiogenic therapy. The overall survival of patients stratified by PWI-based group and gene expression-based subtype was visualized using a boxplot. All statistical analyses were performed using R (version 3.3).

### Survival Analysis of IDH1 Mutation and MGMT Promoter Methylation Co-Variates

The IDH1 mutation status and MGMT promoter methylation status were known for 38 and 8 patients of the 48 patients in the TCGA cohort, respectively, of which 1 patient was mutant in IDH1 mutation and 2 patients harbored MGMT promoter methylation (Table 3). More specifically, one patient (TCGA-06-0128) in PWI-based Cluster I had both the IDH1 mutation and MGMT promoter methylation. The other patient (TCGA-06-0119) with MGMT promoter methylation was also found in Cluster I. Neither IDH1 mutation (log-rank p=0.86) nor MGMT promoter methylation (log-rank p=0.99) was significantly associated with better overall survival, likely due to the small numbers of patients with the information available. The IDH1 mutation status was not available for patients in the MC cohort, while the MGMT promoter methylation status was known for 40 patients (Table 3). Univariate Cox survival analysis showed that the MGMT promoter methylation status was associated with a trend toward decreased risk of death, but the effect was not significant (log-rank p=0.074, HR=0.39).

TABLE 3
Summary of IDH1 mutation status and MGMT promoter
methylation status for the two cohorts.

| | TCGA cohort | | | MC cohort | | |
|---|---|---|---|---|---|---|
| | Cluster I | Cluster II | Whole | Cluster I | Cluster II | Whole |
| IDH1 mutation | 1/25 (6) | 0/13 (4) | 1/38 (10) | NA | NA | NA |
| N/Total available N | | | | | | |
| (Missing N) | | | | | | |
| MGMT promoter | 2/5 (26) | 0/3 (14) | 2/8 (40) | 14/25 (10) | 8/15 (19) | 22/40 (29) |
| methylation | | | | | | |
| N/Total available N | | | | | | |
| (Missing N) | | | | | | |

NA: not available.

### Anti-Angiogenic Chemotherapy Regimen

The first line treatment post-surgery at MC was concurrent chemo-radiation with temozolomide (TMZ), followed by monthly TMZ cycles until the patient showed progression on subsequent Mill scans. Once progression was noted, options included repeat surgery, adding Avastin® to TMZ while continuing TMZ, or considering clinical trials. The decisions for instituting Avastin® was based on a number of variables, including patient preference, the presence of significant brain edema along with the tumor progression (Avastin® helps resolve the brain edema), progression of tumor into non-surgical regions, the lack of eligibility of the patient for clinical trials, etc.

Patients treated without anti-angiogenic therapies were those who were not given anti-angiogenic drugs as part of the chemotherapy regimen at any time of the treatment course.

### Molecular Pathway Analysis

Gene set enrichment analysis (GSEA, MIT) was performed to identify up-regulated gene sets and pathways in the PWI-based clusters. The SAM method was run on microarray gene expression data, with the discovered PWI-based clusters as labels. SAM generated a test statistic for each gene measuring its strength of association with the clusters, which created a ranked list of all genes. Using the gene ontology (GO) as the annotation set and the pre-ranked list of genes, the GSEA algorithm computed significant enrichment in each PWI-based cluster. The top gene sets with FDR q-value<0.05 were reported.

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1: Identification of Subgroups in Newly Diagnosed Glioblastoma Patients Using Quantitative Perfusion Magnetic Resonance Imaging

This example illustrates that robust and clinically relevant subgroups of glioblastoma patients can be identified by leveraging a comprehensive set of perfusion-weighted imaging (PWI) features that characterize both bulk tumor and intra-tumoral heterogeneity.

### Characterization of Patient Cohorts

The median age in the TCGA and MC cohorts was 61 (ranging 30-84) and 60.5 (ranging 21-91) years, respectively. Table 1 shows survival analysis of clinical variables, where known prognostic variables such as Karnofsky performance score (KPS) and multi-centric tumor phenotype are significantly associated with survival in both cohorts, consistent with previous reports (Brennan et al., 2013; Verhaak et al., 2010).

### Unsupervised Clustering Using PWI Features Identifies Two Prognostic Patient Subgroups

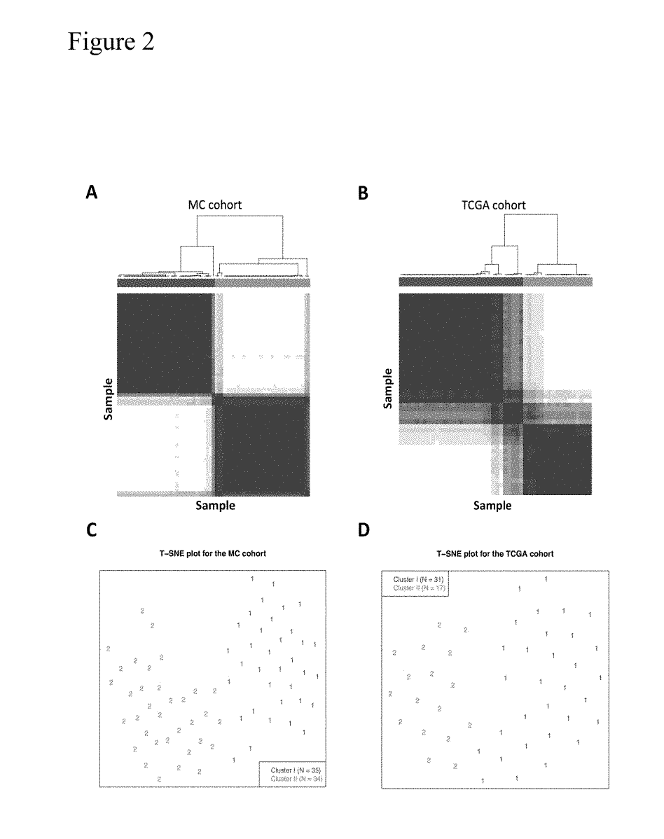

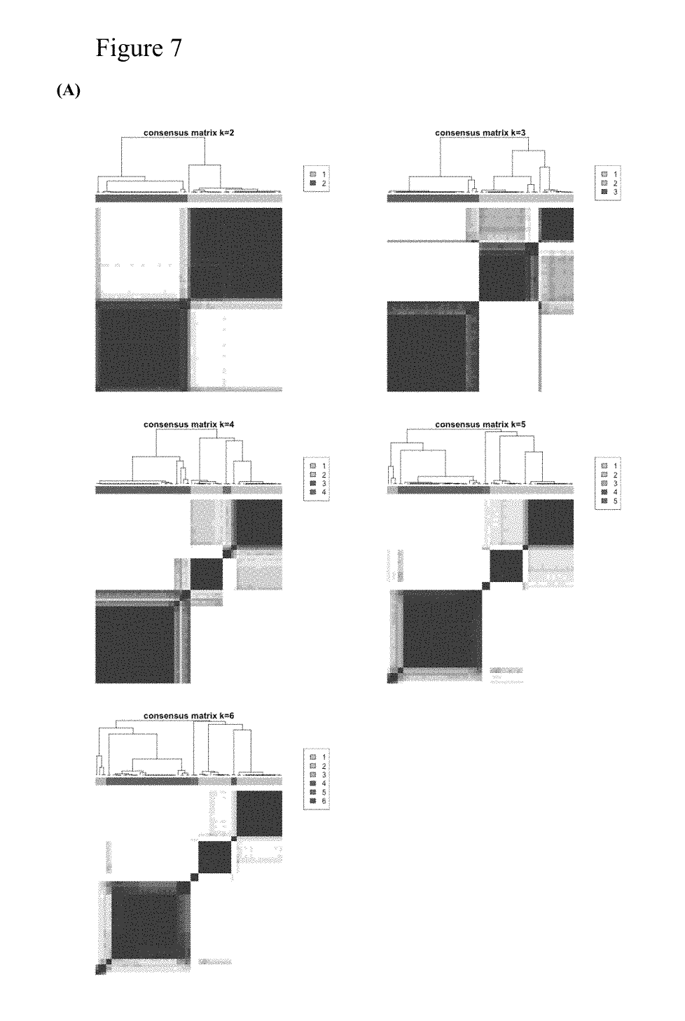

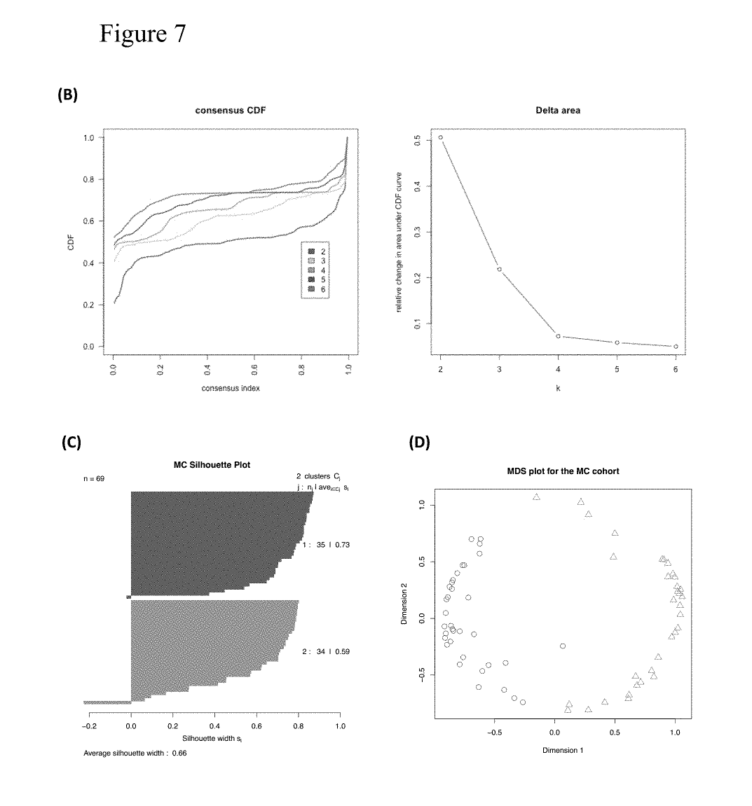

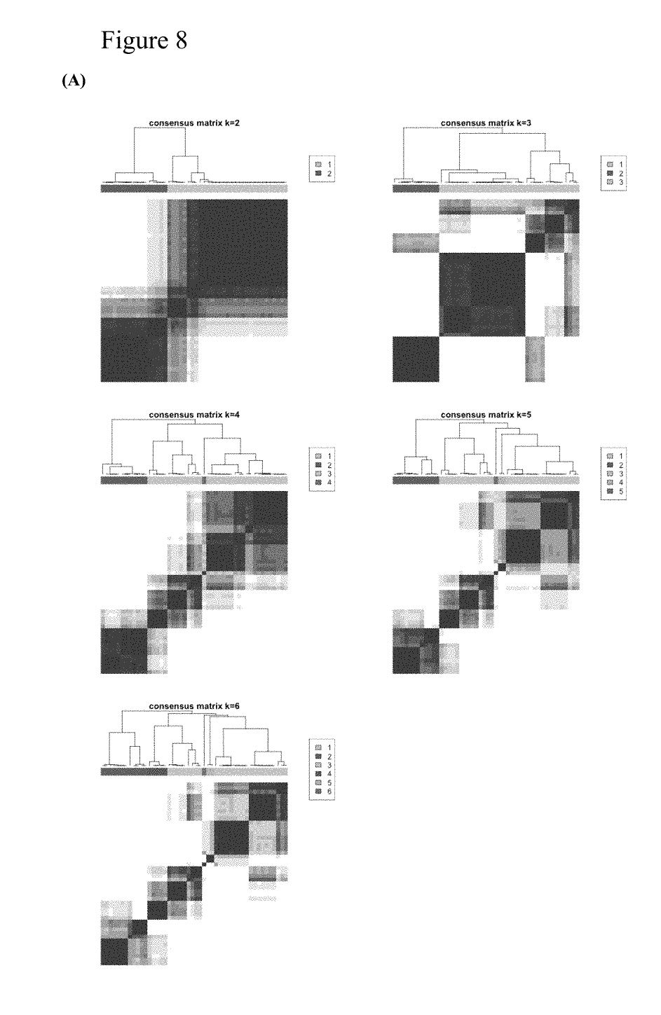

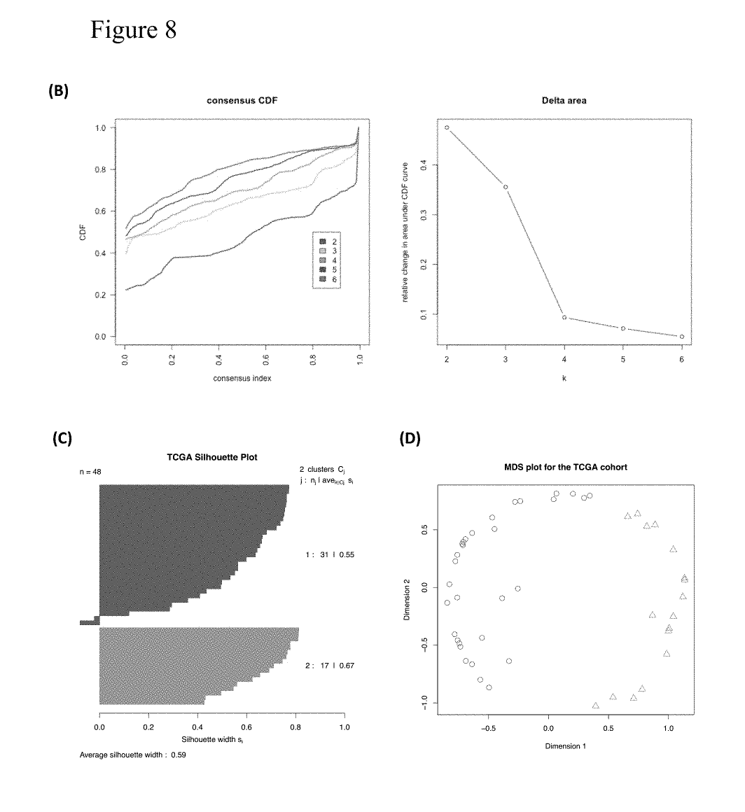

Unsupervised consensus clustering using the 46 PWI features produced 2 clusters in both the TCGA and the MC cohorts, as shown in FIGS. 2A, B. We then computed the overall average silhouette width for the two clusters to evaluate the validity of the number of clusters (Rousseeuw, 1987). The average silhouette widths for the two cohorts were 0.59 and 0.66, providing supporting evidence that the two clusters are robust, as seen in FIG. 7 and FIG. 8. Cluster II forms a distinct cluster from Cluster I, as visualized by the t-distributed stochastic neighbor embedding (T-SNE) plots of both cohorts, as seen in FIGS. 2 C,D.

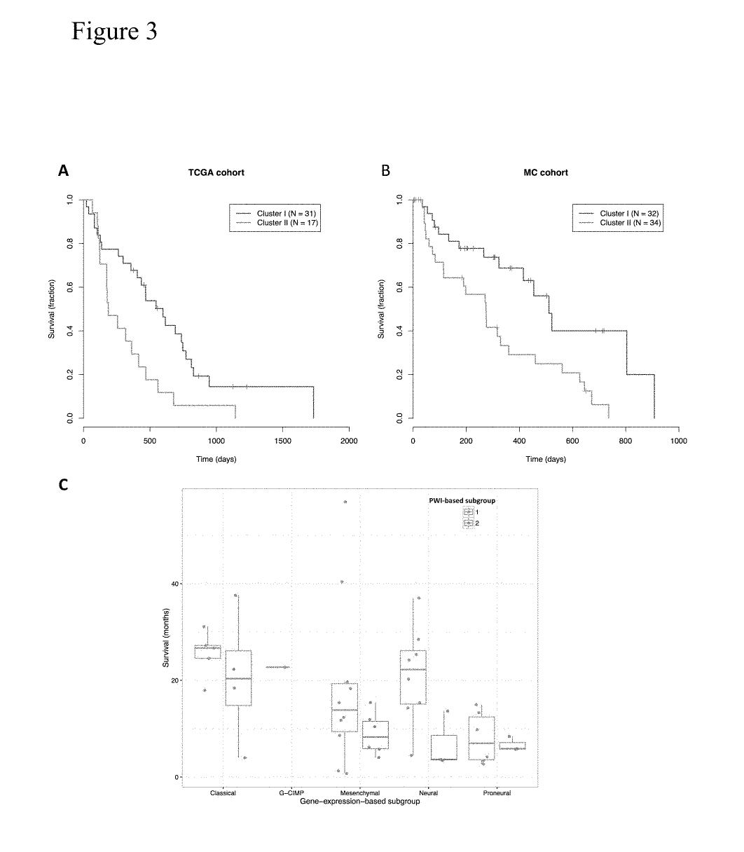

The Kaplan Meier survival analysis showed that Cluster II patients have a significantly worse survival than Cluster I patients in both the TCGA (log-rank p=0.0092, HR=2.30) (FIG. 3A) and MC (log-rank p=0.0041, HR=2.58) cohorts (FIG. 3B). Multivariate Cox analysis showed that this survival difference for Cluster II in TCGA remained significant (log-rank p=0.0033, HR=4.39) after accounting for other clinical variables, including age>60 years, CEL volume, multi-centric tumor phenotype, and KPS, as shown in Table 1. Similarly, the MC cohort confirmed that Group II patients have significantly worse survival (log-rank p=0.0010, HR=3.49), independent of other clinical covariates (Table 1). These results confirms that robust and clinically relevant subgroups could be identified based on a comprehensive set of PWI features.

TABLE 1
Clinical variables and the PWI-based subgroup as covariates in the survival analysis of
GBM patients. Univariate and multivariate Cox proportional hazard models show that PWI-
based subgroups are significantly associated with survival, after accounting for the other
clinical variables in both the TCGA and MC cohorts. Contrast enhancing lesion (CEL) tumor
volume was dichotomized by the median. KPS = Karnofsky performance score. KPS is available
for N = 34 patients in TCGA. Statistically significant values are shown in bold.

| | TCGA | | | | MC cohort | | | |
|---|---|---|---|---|---|---|---|---|
| | Univariate Cox | | Multivariate Cox | | Univariate Cox | | Multivariate Cox | |
| Clinical | HR | p- | HR | p- | HR | p- | HR | p- |
| variable | (95% CI) | value | (95% CI) | value | (95% CI) | value | (95% CI) | value |
| Age at | 1.2 | 0.48 | 1.5 | 0.35 | 2.7 | 0.0044 | 3.4 | 0.0016 |
| initial | [0.7, 2.3] | | [0.6, 3.8] | | [1.4, 5.3] | | [1.6, 7.4] | |
| diagnosis > 60 | | | | | | | | |
| Gender = | 0.7 | 0.39 | — | — | 1.9 | 0.074 | — | — |
| Male | [0.4, 1.4] | | | | [0.9, 3.8] | | | |
| Large | 1.3 | 0.35 | 1.4 | 0.47 | 1.2 | 0.64 | 1.3 | 0.39 |
| CEL | [0.7, 2.5] | | [0.6, 3.3] | | [0.6, 2.3] | | [0.7, 2.6] | |
| volume | | | | | | | | |
| (cm³) | | | | | | | | |
| Multi- | 3.0 | 0.019 | 0.5 | 0.45 | 2.1 | 0.048 | 1.9 | 0.12 |
| centric | [1.2, 7.5] | | [0.07, 3.3] | | [1.0, 4.4] | | [0.8, 4.3] | |
| tumor | | | | | | | | |
| phenotype | | | | | | | | |
| KPS < 80 | 3.1 | 0.0043 | 3.9 | 0.0078 | 2.8 | 0.0017 | 3.0 | 0.0026 |
| | [1.4, 6.8] | | [1.4, 10.7] | | [1.5, 5.4] | | [1.5, 6.2] | |
| PWI-based | 2.3 | 0.0092 | 4.4 | 0.0033 | 2.6 | 0.0041 | 3.5 | 0.0010 |
| subgroup == 2 | [1.2, 4.4] | | [1.6, 11.8] | | [1.3, 5.1] | | [1.7, 7.4] | |

Corroborating with the results obtained from all patients, the PWI-based Cluster II was associated with worse survival than Cluster I consistently across different gene expression-based subtypes, most prominently in the Neural, Classical and Mesenchymal subtypes, as shown in FIG. 3C. Also, the non-G-CIMP, Proneural subtype (log-rank p=0.0053, HR=4.6) was significantly correlated with worse survival than the other subtypes, as shown in Table 4. The multivariate Cox analysis showed that both the PWI-based Cluster II and the gene expression-based non-G-CIMP Proneural subtype were significant indicators of poor prognosis, as described in Tables 4 and 5.

TABLE 4
Cox survival analysis of gene expression-based and PWI-based
subgroups (overall model log-rank p = 0.0029). The Classical
subtype was used as reference. Statistically significant values are
shown in bold.

| | Univariate Cox | | Multi-variate Cox | |
|---|---|---|---|---|
| | HR [95% CI] | p-value | HR [95% CI] | p-value |
| PWI-based | 2.3 [1.2, 4.4] | 0.0092 | 2.9 [1.4, 6.2] | 0.0042 |
| subgroup == II | | | | |
| Gene-expression- | | | | |
| based subgroup | | | | |
| Classical (N = 8) | — | — | — | — |
| G-CIMP (N = 1) | 1.1 [0.1, 9.1] | 0.90 | 2.0 [0.2, 16.9] | 0.53 |
| Mesenchymal | 1.6 [0.6, 3.8] | 0.33 | 2.1 [0.8, 5.3] | 0.12 |
| (N = 11) | | | | |
| Neural (N = 7) | 1.2 [0.5, 3.2] | 0.64 | 1.9 [0.7, 5.4] | 0.19 |
| Proneural (N = 7) | 4.6 [1.6, 13.6] | 0.0053 | 6.3 [2.0, 19.8] | 0.0016 |

TABLE 5
Full multivariate Cox model (overall model log-rank p =
0.002867) for the TCGA cohort. Gender is excluded in the
full model, as it is not a clinical prognostic covariate.
KPS is available for N = 34 patients in TCGA. Statistically
significant values are shown in bold.

| | HR [95% CI] | p-value |
|---|---|---|
| Age at initial diagnosis >60 | 1.2 [0.4, 3.3] | 0.72 |
| Large CEL volume (cm³) | 1.4 [0.5, 3.5] | 0.51 |
| Multi-centric tumor phenotype | 0.2 [0.02 2.5] | 0.23 |
| KPS <80 | 8.6 [2.4, 30.1] | 0.00077 |
| Gene-expression-based subgroup | | |
| Classical (N = 9) | — | — |
| G-CIMP (N = 1) | 4.7 [0.4, 62.0] | 0.24 |
| Mesenchymal (N = 16) | 3.3 [0.8, 14.0] | 0.11 |
| Neural (N = 11) | 2.3 [0.6, 8.5] | 0.20 |
| Proneural (N = 9) | 21.4 [4.0, 114.9] | 0.00036 |
| PWI-based subgroup == II | 9.1 [2.6, 31.9] | 0.00057 |

### Example 2

### Defining the Molecular Profiles of Subgroups Identified in Glioblastoma Patients Based on Intra-Tumor Perfusion-Weighted Imaging (PWI) Features

This example illustrates that intra-tumor perfusion-weighted imaging features, obtained from molecular profiling of the patient subgroups, were more informative in detecting patient subgroups than summary perfusion-weighted imaging features.

Cluster II Patients are Associated with High Intra-Tumor PWI Features

Summary PWI features alone extracted from the whole enhancing tumor, including mean, median, kurtosis, skewness, max and variance, that were obtained in Example 1, were not consistently associated with the discovered clusters in the two cohorts, confirming previous reports (Jain et al., 2013). Moreover, univariate survival analysis revealed that none of the 6 summary perfusion features as a continuous variable was significantly associated with overall survival in either cohort. In the MC cohort, the binary rCBVₘₑₐₙ, rCBV_{median}, and rCBV_{variance} dichotomized by the median of each feature was each significantly correlated with survival (log-rank p-values<0.05). After adjusting for multiple hypothesis testing, high rCBV_{median} remained significantly prognostic (HR=2.55, log-rank p=0.0064, adjusted p=0.029). In the TCGA cohort, on the other hand, high rCBVmean was significantly correlated with poor survival before multiple hypothesis correction (HR=2.00, log-rank p=0.027). After multiple hypothesis correction, none of the summary perfusion features in TCGA was significantly associated with survival.

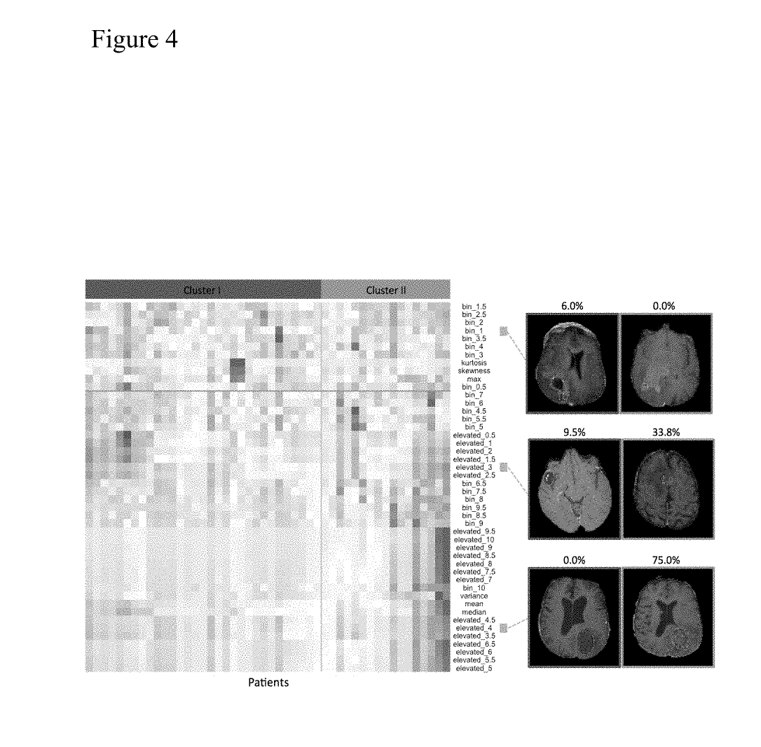

As evident from FIG. 4, the heatmaps of the PWI features revealed the difference between the two clusters of patients, with most histogram-based regional PWI features in Cluster II being larger than those in Cluster I. In FIG. 4, as shown in the example images of three PWI features in the two clusters, Cluster II in TCGA was positively associated with a larger number of voxels at a low to medium cutoff, such as rCBV_{elevated}_{_}₃ and rCBV_{elevated}_{_}₄, corresponding to a large fraction of voxels with values greater than the cutoff, which were colored in red for visualization.

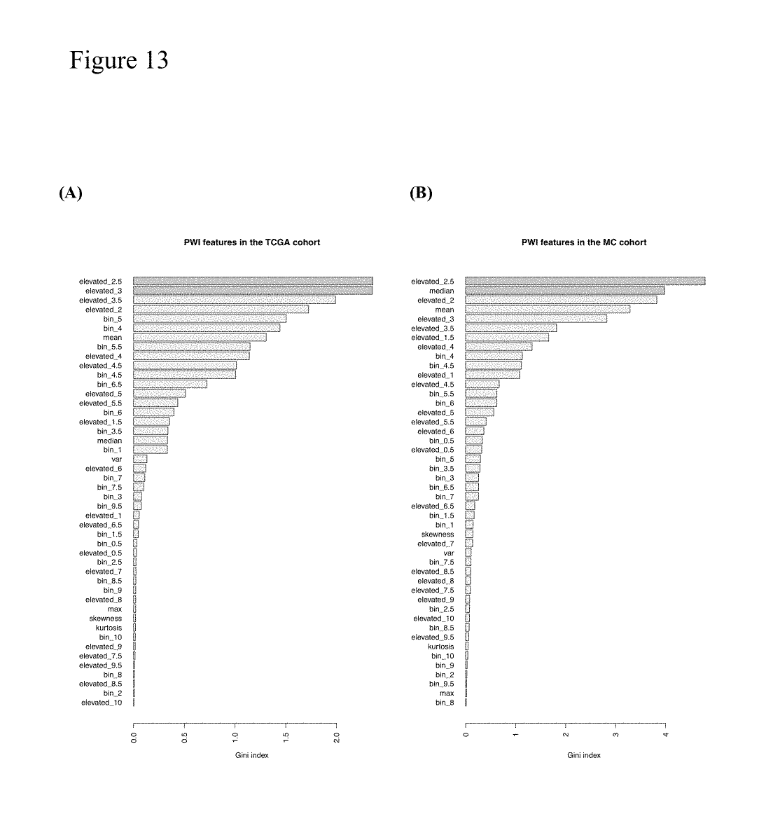

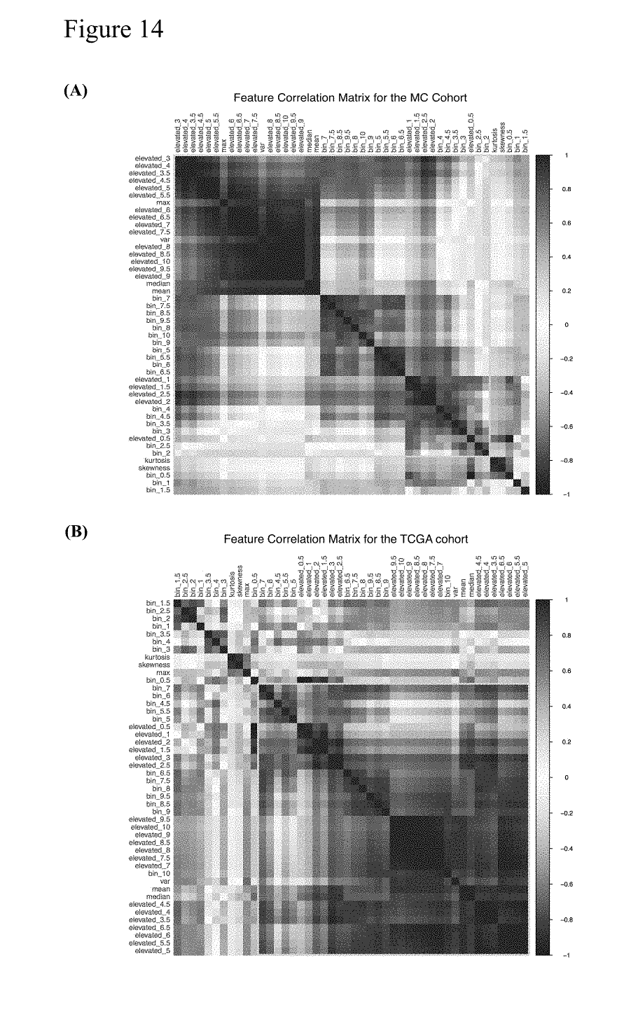

A random forest trained on the TCGA cohort confirmed that rCBV_{elevated} features at low to medium cutoffs were predictive of the two clusters, as seen in FIG. 13A. These PWI imaging feature patterns that are characteristics of the two clusters were similarly observed in the MC cohort, see FIG. 13B. Since many of these PWI features were highly correlated (redundant) (FIG. 14), the recursive feature elimination algorithm selected a handful of features that were predictive of the two clusters, including rCBV_{elevated}_{_}_{2.5} and rCBV_{elevated}_{_}₃ for the TCGA cohort, and rCBV_{elevated}_{_}_{2.5} and rCBV_{median} for the MC cohort, as seen in FIG. 13.

To validate the generalizability of the significant PWI features associated with the clusters to unseen cases, the random forest classifier that was constructed with the MC cohort was then used to classify patients of the TCGA cohort into two groups. Comparing the classifier-based stratification with the unsupervised clustering approach above, the accuracy of predicting the TCGA cohort using a model trained on all PWI features in the MC cohort was 95.8% (46/48), and the model trained on the selected subset of features was 97.9% (47/48).

The classifier-based stratification trained on the MC cohort remained significantly associated with survival in TCGA (log-rank p=0.030, HR=1.98). Similarly, the classification accuracy was 92.8% (64/69) for training on all features in TCGA and predicting on the MC cohort, and was 94.2% (65/69) for training on the selected subset of features in TCGA. The classifier-based stratification of the MC cohort trained on TCGA was significant in correlating with survival (log-rank p=0.012, HR=2.26).

### Example 3

In this study, the treatment response of the in Examples 1 and 2 classified patient subgroups to anti-angiogenic therapy was assessed.

### PWI-Based Cluster II Patients are Enriched for Angiogenesis

A gene set enrichment analysis (GSEA) was employed to identify molecular activities that are different between the two clusters (Subramanian et al., 2005). A total of 13 gene sets, including angiogenesis signaling pathway, vasculature development, and response to hypoxia, were found to be significantly enriched in Cluster II compared to Cluster I (FDR p<0.05) (Table 2). Shared genes contributing to the core enrichment of both the hypoxia signaling and the angiogenesis pathways consisted of angiogenin (ANG), VEGF A, and transforming growth factor beta 2 (TGFB2, also called glioblastoma-derived T-cell suppressor factor). Up-regulation of angiogenesis pathways found in Cluster II suggests the potential for treatment efficacy using anti-angiogenic therapy in this subgroup of patients.

TABLE 2
Top pathways enriched in Cluster II. GSEA analysis revealed
that Response to hypoxia, Angiogenesis, and Vasculature
development pathways were enriched in Cluster II.

| | GENE SET | FDR q-val |
|---|---|---|
| 1 | I KAPPAB KINASE NF KAPPAB CASCADE | 0.0052 |
| 2 | CYTOKINE ACTIVITY | 0.0093 |
| 3 | RESPONSE TO HYPOXIA | 0.010 |
| 4 | REGULATION OF I KAPPAB KINASE NF KAPPAB | 0.012 |
| | CASCADE | |
| 5 | ANATOMICAL STRUCTURE FORMATION | 0.020 |
| 6 | HYDROLASE ACTIVITY HYDROLYZING O | 0.020 |
| | GLYCOSYL COMPOUNDS | |
| 7 | ANGIOGENESIS | 0.020 |
| 8 | OXIDOREDUCTASE ACTIVITY GO 0016705 | 0.021 |
| 9 | VASCULATURE DEVELOPMENT | 0.021 |
| 10 | POSITIVE REGULATION OF I KAPPAB KINASE NF | 0.021 |
| | KAPPAB CASCADE | |
| 11 | ER GOLGI INTERMEDIATE COMPARTMENT | 0.021 |
| 12 | OXIDOREDUCTASE ACTIVITY ACTING ON THE | 0.022 |
| | CH CH GROUP OF DONORS | |
| 13 | RESPONSE TO WOUNDING | 0.023 |

PWI-Based Cluster II Patients Given Anti-Angiogenic Treatment have Better Survival

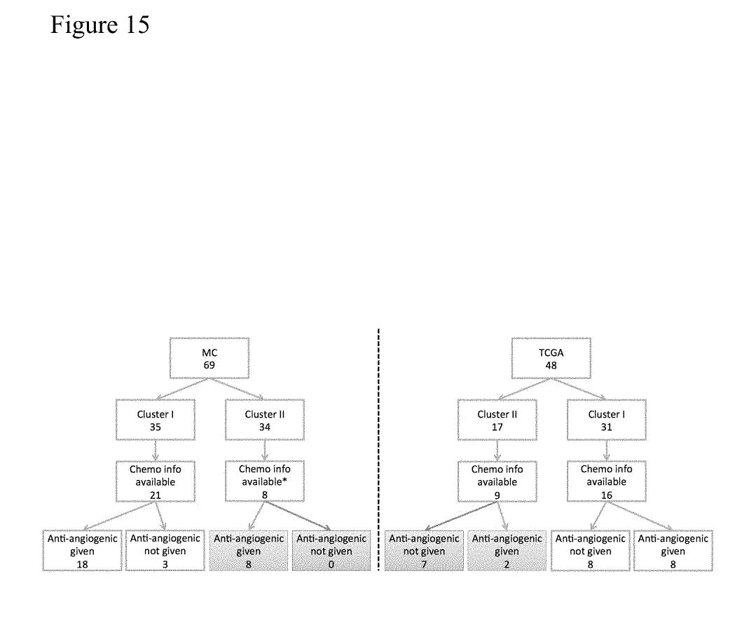

We next evaluated whether the PWI-based quantitative imaging features can be used as biomarkers to predict treatment response to anti-angiogenic therapy in GBM patients, based on identifying the cluster to which they belong. Because chemotherapy treatment information was only available for a subset of patients in both of our cohorts (FIG. 15), we combined patients with chemotherapy information from both cohorts to increase statistical power. Anti-angiogenic treatment did not prolong overall survival in all patients as a single group (log-rank p=0.15, HR=0.59), consistent with results reported in a recent large-scale clinical trial (Gilbert et al., 2014).

In the Cluster II patients who were predicted to respond to anti-angiogenic treatment from both cohorts, those treated with anti-angiogenic therapies (median survival: 552.5 days) had significantly longer survival than those who were not given the anti-angiogenic therapy (median survival: 178 days) (log-rank p=0.022, HR=0.28) (FIG. 5), with a median survival difference of more than 1 year (374.5 days). In contrast, anti-angiogenic treatment (N=26/37) did not confer survival advantage in the Cluster I patients (log-rank p=0.77, HR=0.86), as might be predicted from the differential PWI feature and molecular analyses. More specifically, the median survival for patients treated with and without anti-angiogenic therapy in Cluster I was 439 and 546 days, respectively.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Barajas R F, Jr., Phillips J J, Parvataneni R, et al. Regional variation in histopathologic features of tumor specimens from treatment-naive glioblastoma correlates with anatomic and physiologic M R Imaging. Neuro Oncol. 2012; 14(7):942-954.
Barajas R F, Jr., Cha S. Benefits of dynamic susceptibility-weighted contrast-enhanced perfusion MRI for glioma diagnosis and therapy. CNS Oncol. 2014; 3(6):407-419.
Bolstad B M, Irizarry R A, Astrand M, Speed T P. A comparison of normalization methods for high density oligonucleotide array data based on variance and bias. Bioinformatics. 2003; 19(2): 185-193.
Boxerman J L, Schmainda K M, Weisskoff R M. Relative cerebral blood volume maps corrected for contrast agent extravasation significantly correlate with glioma tumor grade, whereas uncorrected maps do not. AJNR Am J Neuroradiol 2006; 27(4):859-867.
Bradburn M J, Clark T G, Love S B, Altman D G. Survival Analysis Part II: Multivariate data analysis—an introduction to concepts and methods. Br J Cancer 2003; 89:431-436.
Brennan C W, Verhaak R G, McKenna A, et al. The somatic genomic landscape of glioblastoma. Cell. 2013; 155(2):462-477.
Chinot O L, Wick W, Mason W, et al. Bevacizumab plus radiotherapy-temozolomide for newly diagnosed glioblastoma. N Engl J Med. 2014; 370(8):709-722.
Clark K, Vendt B, Smith K, et al. The Cancer Imaging Archive (TCIA): maintaining and operating a public information repository. J Digit Imaging. 2013; 26(6):1045-1057.
Cox D R. Regression models and life tables. J R Statist Soc B. 1972. 34:187-220.
Cox T F, Cox M A A. Multidimensional scaling: CRC press; 2000.
Davnall F, Yip C S, Ljungqvist G, et al. Assessment of tumor heterogeneity: an emerging imaging tool for clinical practice? Insights Imaging. 2012; 3(6):573-589.
Fedorov A, Beichel R, Kalpathy-Cramer J, et al. 3D Slicer as an image computing platform for the Quantitative Imaging Network. Magn Reson Imaging. 2012; 30(9):1323-1341.
Friedman H S, Prados M D, Wen P Y, et al. Bevacizumab alone and in combination with irinotecan in recurrent glioblastoma. J Clin Oncol. 2009; 27(28):4733-4740.
Gerlinger M, Rowan A J, Horswell S, et al. Intratumor heterogeneity and branched evolution revealed by multiregion sequencing. N Engl J Med. 2012; 366(10):883-892.
Gevaert O, Mitchell L A, Achrol A S, et al. Glioblastoma multiforme: exploratory radiogenomic analysis by using quantitative image features. Radiology. 2014; 273(1):168-174.
Gilbert M R, Dignam J J, Armstrong T S, et al. A randomized trial of bevacizumab for newly diagnosed glioblastoma. N Engl J Med. 2014; 370(8):699-708.
Hakyemez B, Erdogan C, Bolca N, Yildirim N, Gokalp G, Parlak M. Evaluation of different cerebral mass lesions by perfusion-weighted MR imaging. J Magn Reson Imaging. 2006; 24(4):817-824.
Hu L S, Baxter L C, Pinnaduwage D S, et al. Optimized preload leakage-correction methods to improve the diagnostic accuracy of dynamic susceptibility-weighted contrast-enhanced perfusion MR imaging in posttreatment gliomas. AJNR Am J Neuroradiol. 2010; 31(1):40-48.
Hu L S, Eschbacher J M, Heiserman J E, et al. Reevaluating the imaging definition of tumor progression: perfusion MRI quantifies recurrent glioblastoma tumor fraction, pseudoprogression, and radiation necrosis to predict survival. Neuro Oncol. 2012; 14(7):919-930.
Itakura H, Achrol A S, Mitchell L A, et al. Magnetic resonance image features identify glioblastoma phenotypic subtypes with distinct molecular pathway activities. Sci Transl Med. 2015; 7(303):303ra138.
Jain R, Poisson L, Narang J, et al. Genomic mapping and survival prediction in glioblastoma: molecular subclassification strengthened by hemodynamic imaging biomarkers. Radiology. 2013; 267(1):212-220.
Johnson H, Harris G, Williams K. BRAINSFit: mutual information rigid registrations of whole-brain 3D images, using the insight toolkit. 2007.
Kreisl T N, Kim L, Moore K, et al. Phase II trial of single-agent bevacizumab followed by bevacizumab plus irinotecan at tumor progression in recurrent glioblastoma. J Clin Oncol. 2009; 27(5): 740-745.
Kuhn M. Caret package. Journal of Statistical Software. 2008; 28(5).
Liaw A, Wiener M. Classification and regression by randomForest. R news. 2002; 2(3):18-22.
Liu T T, Achrol A S, Mitchell L A, et al. Computational Identification of Tumor Anatomic Location Associated with Survival in 2 Large Cohorts of Human Primary Glioblastomas. AJNR Am J Neuroradiol. 2016; 37(4):621-628.
Lu-Emerson C, Duda D G, Emblem K E, et al. Lessons from anti-vascular endothelial growth factor and anti-vascular endothelial growth factor receptor trials in patients with glioblastoma. J Clin Oncol. 2015; 33(10):1197-1213.
Maaten Lvd, Hinton G. Visualizing data using t-SNE. Journal of Machine Learning Research. 2008; 9(November):2579-2605.
Mita M M, Sargsyan L, Mita A C, Spear M. Vascular-disrupting agents in oncology. Expert Opin Invest Drugs 22: 317-328 (2013).
Monk B J, Minion L E, Coleman R L. Anti-angiogenic agents in ovarian cancer: past, present, and future. Ann Oncol 27:i33-i39 (2016).
Monti S, Tamayo P, Mesirov J, Golub T. Consensus clustering: a resampling-based method for class discovery and visualization of gene expression microarray data. Machine learning. 2003; 52(1-2):91-118.
Omuro A, DeAngelis L M. Glioblastoma and other malignant gliomas: a clinical review. JAMA. 2013; 310(17):1842-1850.
Patel A P, Tirosh I, Trombetta J J, et al. Single-cell RNA-seq highlights intratumoral heterogeneity in primary glioblastoma. Science. 2014; 344(6190):1396-1401.
Paulson E S, Schmainda K M. Comparison of dynamic susceptibility-weighted contrast-enhanced M R methods: recommendations for measuring relative cerebral blood volume in brain tumors. Radiology. 2008; 249(2):601-613.
Phillips H S, Kharbanda S, Chen R, Forrest W F, Soriano R H, Wu T D, Misra A, Nigro J M, Colman H, Soroceanu L, Williams P M, Modrusan Z, Feuerstein B G, Aldape K. Molecular subclasses of high-grade glioma predict prognosis, delineate a pattern of disease progression, and resemble stages in neurogenesis. Canc cell. 2006. 9:157-173.
Rousseeuw P J. Silhouettes: a graphical aid to the interpretation and validation of cluster analysis. Journal of computational and applied mathematics. 1987; 20:53-65%@ 0377-0427.
Solt L A & May M J. The IkB kinase complex: master regulator of NF-kB signaling. Immunol Res. 2008. 42:3-18.
Subramanian A, Tamayo P, Mootha V K, et al. Gene set enrichment analysis: a knowledge-based approach for interpreting genome-wide expression profiles. Proc Natl Acad Sci USA. 2005; 102(43):15545-15550.
Tarca A L, Romero R, Draghici S. Analysis of microarray experiments of gene expression profiling. Am J Obstet Gynecol. 2006. 195:373-388.
The Cancer Genome Atlas (TCGA) Research Network. Comprehensive genomic characterization defines human glioblastoma genes and core pathways. Nature. 2008; 455:1061-1068.
Thomas A A, Brennan C W, DeAngelis L M, Omuro A M. Emerging therapies for glioblastoma. JAMA Neurol. 2014; 71(11):1437-1444.
Ucuzian A A, Gassman A A, East A T, Greisler H P. Molecular Mediators of Angiogenesis. J Burn Care Res. 2010. 31:158-176.
Verhaak R G, Hoadley K A, Purdom E, et al. Integrated genomic analysis identifies clinically relevant subtypes of glioblastoma characterized by abnormalities in PDGFRA, IDH1, EGFR, and NF1. Cancer Cell. 2010; 17(1):98-110.
Weller M, Yung W K. Angiogenesis inhibition for glioblastoma at the edge: beyond AVAGlio and RTOG 0825. Neuro Oncol. 2013; 15(8):971.
Yadav M, Jhunjhunwala S, Phung Q T, et al. Predicting immunogenic tumour mutation by combining mass spectrometry and exome sequencing. Nature. 2014; 515(7528):572-576.
Zhang X, Pagel M D, Baker A F, Gillies R J. Reproducibility of magnetic resonance perfusion imaging. PLoS One. 2014; 9(2):e89797.

What is claimed is:

1. A computer-implemented method for non-invasively identifying a subject suffering from a brain tumor as susceptible to anti-angiogenic therapy, comprising
determining quantitative image features from tissue of said brain tumor to obtain a phenotypic characterization of blood perfusion of said tumor and intra-tumor heterogeneity;
optionally determining an intra-tumor specific molecular profile of said brain tumor;
combining information from said image features and optionally from said molecular profile to determine said subject's tumor angiogenesis profile; and
comparing said subject's tumor angiogenesis profile with a reference angiogenesis profile,
wherein a deviating profile for said subject relative to said reference profile identifies said subject as susceptible to anti-angiogenic therapy.

2. The method according to claim 1, wherein the brain tumor is glioblastoma.

3. The method according to claim 1, wherein the quantitative image features are determined by measuring perfusion-weighted image data using magnetic resonance imaging.

4. The method according to claim 1, wherein said optional molecular profile is determined by contacting a biological sample from said subject with reagents suitable for detecting expression levels of at least one gene product related to at least one of angiogenesis, response to hypoxia, and vasculature development, and
detecting levels of expression of at least one gene product related to at least one of angiogenesis, response to hypoxia, and vasculature development,
wherein a deviating level of expression of said at least one gene product related to at least one of angiogenesis, response to hypoxia, and vasculature development in comparison to a reference may indicate an increase in intra-tumor angiogenesis pathways.

5. The method according to claim 1, wherein a deviating profile that is indicative of said subject's susceptibility to anti-angiogenic therapy is characterized by an increase in intra-tumor angiogenesis pathways and elevated quantitative image features.

6. The method according to claim 4, wherein said molecular profile is determined by detecting the expression level of gene products from at least 1, 2, 3, 4, 5, 6, 7, 8, 9, 10, 11, 12, 13, 14, 15, 16, 17, 18, 19, 20, 30, 40, 50, 60 gene(s) selected from the group consisting of ACVRL1, AGGF1, ALAS2, AMOT, ANG, ANGPTL3, ANGPTL4, ARNT2, ATPIF1, BNIP3, BTG1, C1GALT1, CANX, CCM2, CD24, CDH13, CHRNA4, CHRNA7, CHRNB2, CLDN3, COL4A2, COL4A3, CREBBP, CUL7, CXCR4, EGF, EGFL7, EGLN1, EGLN2, EMCN, EP300, EPAS1, EPGN, ERAP1, FOXC2, FOXO4, GLMN, HIF1A, HSP90B1, HTATIP2, IL17F, IL18, IL8, MYH9, MT3, NARFL, NCL, NF1, NOTCH4, NPPB, NPR1, PDIA2, PDPN, PF4, PLG, PLOD1, PLOD2, PML, PROK2, RASA1, RHOB, RNH1, ROBO4, RUNX1, SCG2, SERPINF1, SHH, SMAD3, SMAD4, SPHK1, SPINK5, STAB1, TGFB2, THY1, TNFSF12, TNNI3, VEGF-A.

7. The method according to claim 4, wherein said molecular profile is determined by detecting the expression level of gene products from at least 1, 2, 3, 4, 5, 6, 7, 8, 9, 10 gene(s) selected from the group consisting of VEGF-A, PLOD2, PLOD1, HSP90B1, ANG, EGLN1, BNIP3, EPAS1, TGFB2, CXCR4.

8. The method according to claim 4, wherein said molecular profile is determined by detecting the expression level of gene products from at least 1, 2, 3, 4, 5, 6, 7, 8, 9, 10, 11, 12, 13, 14, 15 gene(s) selected from the group consisting of VEGF-A, IL8, RNH1, CANX, ANG, ANGPTL4, COL4A2, MYH9, RUNX1, PF4, EGF, TGFB2, NPPB, AGGF1, NOTCH4.

9. The method according to claim 4, wherein said molecular profile is determined by detecting the expression level of gene products from at least 1, 2, 3, 4, 5, 6, 7, 8, 9, 10, 11, 12, 13, 14, 15, 16, 17 gene(s) selected from the group consisting of VEGF-A, IL8, RNH1, CANX, ANG, ANGPTL4, COL4A2, PDPN, MYH9, RUNX1, PF4, EGF, CUL7, TGFB2, NPPB, AGGF1, NOTCH4.

10. The method according to any of claims 4, 6, 7, 8, and 9, wherein said gene product is a messenger RNA.

11. The method according to any of claims 4, 6, 7, 8, and 9, wherein said gene product is a protein.

12. A method for selecting a treatment for a subject suffering from a brain tumor who may be susceptible to anti-angiogenic therapy, comprising
determining quantitative image features from tissue of said brain tumor to obtain a phenotypic characterization of blood perfusion of said tumor and intra-tumor heterogeneity;
optionally determining an intra-tumor specific molecular profile of said brain tumor;
combining information from said image features and optionally from said molecular profile to determine said subject's tumor angiogenesis profile;
comparing said subject's tumor angiogenesis profile with a reference angiogenesis profile, wherein a deviating profile for said subject relative to said reference profile identifies said subject as susceptible to anti-angiogenic therapy; and
selecting for said subject, if found susceptible to anti-angiogenic therapy, an anti-angiogenic treatment in addition to chemotherapy and/or radiation therapy.

13. The method according to claim 12, wherein the brain tumor is glioblastoma.

14. The method according to claim 12, wherein the quantitative image features are determined by measuring perfusion-weighted image data using magnetic resonance imaging.

15. The method according to claim 12, wherein said optional molecular profile is determined by contacting a biological sample from said subject with reagents suitable for detecting expression levels of at least one gene product related to at least one of angiogenesis, response to hypoxia, and vasculature development, and
detecting levels of expression of at least one gene product related to at least one of angiogenesis, response to hypoxia, and vasculature development
wherein a deviating level of expression of said at least one gene product related to at least one of angiogenesis, response to hypoxia, and vasculature development in comparison to a reference may indicate an increase in intra-tumor angiogenesis pathways.

16. The method according to claim 12, wherein a deviating profile that is indicative of said subject's susceptibility to anti-angiogenic therapy is characterized by an increase in intra-tumor angiogenesis pathways and elevated quantitative image features.

17. The method according to claim 15, wherein said molecular profile is determined by detecting the expression level of gene products from at least 1, 2, 3, 4, 5, 6, 7, 8, 9, 10, 11, 12, 13, 14, 15, 16, 17, 18, 19, 20, 30, 40, 50, 60 gene(s) selected from the group consisting of ACVRL1, AGGF1, ALAS2, AMOT, ANG, ANGPTL3, ANGPTL4, ARNT2, ATPIF1, BNIP3, BTG1, C1GALT1, CANX, CCM2, CD24, CDH13, CHRNA4, CHRNA7, CHRNB2, CLDN3, COL4A2, COL4A3, CREBBP, CULT, CXCR4, EGF, EGFL7, EGLN1, EGLN2, EMCN, EP300, EPAS1, EPGN, ERAP1, FOXC2, FOXO4, GLMN, HIF1A, HSP90B1, HTATIP2, IL17F, IL18, IL8, MYH9, MT3, NARFL, NCL, NF1, NOTCH4, NPPB, NPR1, PDIA2, PDPN, PF4, PLG, PLOD1, PLOD2, PML, PROK2, RASA1, RHOB, RNH1, ROBO4, RUNX1, SCG2, SERPINF1, SHH, SMAD3, SMAD4, SPHK1, SPINK5, STAB1, TGFB2, THY1, TNFSF12, TNNI3, VEGF-A.

18. The method according to claim 15, wherein said molecular profile is determined by detecting the expression level of gene products from at least 1, 2, 3, 4, 5, 6, 7, 8, 9, 10 gene(s) selected from the group consisting of VEGF-A, PLOD2, PLOD1, HSP90B1, ANG, EGLN1, BNIP3, EPAS1, TGFB2, CXCR4.

19. The method according to claim 15, wherein said molecular profile is determined by detecting the expression level of gene products from at least 1, 2, 3, 4, 5, 6, 7, 8, 9, 10, 11, 12, 13, 14, 15 gene(s) selected from the group consisting of VEGF-A, IL8, RNH1, CANX, ANG, ANGPTL4, COL4A2, MYH9, RUNX1, PF4, EGF, TGFB2, NPPB, AGGF1, NOTCH4.

20. The method according to claim 15, wherein said molecular profile is determined by detecting the expression level of gene products from at least 1, 2, 3, 4, 5, 6, 7, 8, 9, 10, 11, 12, 13, 14, 15, 16, 17 gene(s) selected from the group consisting of VEGF-A, IL8, RNH1, CANX, ANG, ANGPTL4, COL4A2, PDPN, MYH9, RUNX1, PF4, EGF, CULT, TGFB2, NPPB, AGGF1, NOTCH4.

21. The method according to any of claims 15, 17, 18, 19, and 20, wherein said gene product is a messenger RNA.

22. The method according to any of claims 15, 17, 18, 19, and 20, wherein said gene product is a protein.
